(19) United States
(12) Patent Application Publication
(10) Pub. No.: US 2020/0121666 A1
(43) Pub. Date: Apr. 23, 2020

(54) TAU PHOSPHORYLATION INHIBITORS AND METHODS FOR TREATING OR PREVENTING ALZHEIMER'S DISEASE

(71) Applicants: THE METHODIST HOSPITAL SYSTEM, Houston, TX (US); THE GENERAL HOSPITAL CORPORATION, Boston, MA (US)

(72) Inventors: Stephen T.C. WONG, Missouri City, TX (US); Xiaofeng XIA, Houston, TX (US); Xiaoyun XU, Pearland, TX (US); Doo Yeon KIM, Charlestown, MA (US); Rudolph E. TANZI, Milton, MA (US)

(21) Appl. No.: 16/619,528

(22) PCT Filed: Jun. 5, 2018
(86) PCT No.: PCT/US2018/036031

§ 371 Date: Dec. 5, 2019

Related U.S. Application Data
(60) Provisional application No. 62/515,132, filed on Jun. 5, 2017.
(60) Provisional application No. 62/515,154, filed on Jun. 5, 2017.

(51) Int. Cl. A61K 31/4439; A61K 31/4706; A61K 31/4184; A61K 31/66; A61K 31/4045; A61K 31/437; A61K 31/52; A61K 31/415; A61K 31/502; A61K 31/198; A61K 31/506; A61K 31/35; A61K 31/5415

(52) U.S. Cl. CPC A61K 31/4439; A61K 31/4706; A61K 31/4184; A61K 31/66; A61K 31/4045; A61K 31/437; A61K 45/06; A61K 31/415; A61K 31/502; A61K 31/198; A61K 31/506; A61K 31/35; A61K 31/5415; A61K 31/52

(57) ABSTRACT

The present disclosure relates to compounds that are useful as tau phosphorylation inhibitors. Further disclosed are compounds and methods for treating or preventing Alzheimer's disease.

### CROSS REFERENCE TO RELATED APPLICATIONS

This application claims the benefit of U.S. Provisional Patent Application Ser. No. 62/515,132 filed Jun. 5, 2017 and U.S. Provisional Patent Application Ser. No. 62/515,154 filed Jun. 5, 2017, which are expressly incorporated herein by reference in their entirety.

### FIELD

The present disclosure relates to compounds that are useful as tau phosphorylation inhibitors. Further disclosed are compounds and methods for treating or preventing Alzheimer's disease.

### BACKGROUND

Alzheimer's disease (AD) currently afflicts 5.3 million people in the United States alone. Increasing evidence suggests that tau pathology underlies the learning and memory deficit in Alzheimer's disease. Tau pathology is characterized by the hyperphosphorylation of the microtubule associated protein tau, leading to its misfolding and aggregation in neuronal cells. Compounds preventing or reversing tau protein hyperphosphorylation therefore hold potential for the treatment of AD.

Despite many years of research, outside of symptomatic treatment, no clear therapeutic options are available for Alzheimer's disease (AD) patients. Conventional drug discovery paradigms to identify new therapeutic candidates are ill-equipped to combat a disease as complex as AD. To date the identification of such compounds has been hampered by the lack of a faithful in vitro cellular model and effective high-throughput screening method.

The compounds, compositions, and methods disclosed herein address these and other needs.

### SUMMARY

Disclosed herein are compounds and methods for the treatment and/or prevention of Alzheimer's disease. To develop a high-throughput in vitro cellular model, a modified neural stem cell model was used which gradually develops tau pathology during culture. Using the modified high-throughput in vitro cellular model, several compounds were identified that regulate levels of tau phosphorylation and are useful for treating or preventing Alzheimer's disease.

In one aspect, disclosed herein is a method for treating or preventing Alzheimer's disease comprising administering to a subject in need thereof a therapeutically effective amount of a compound selected from the following:

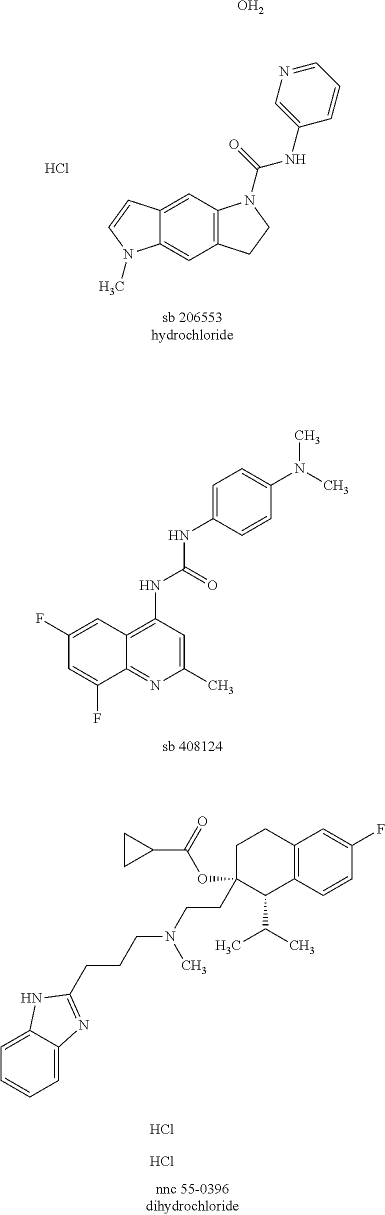

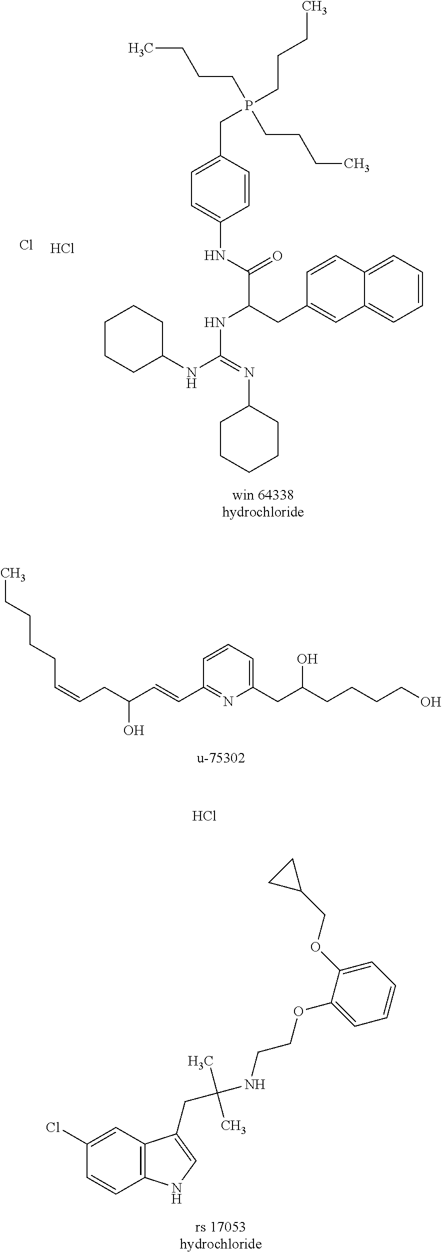

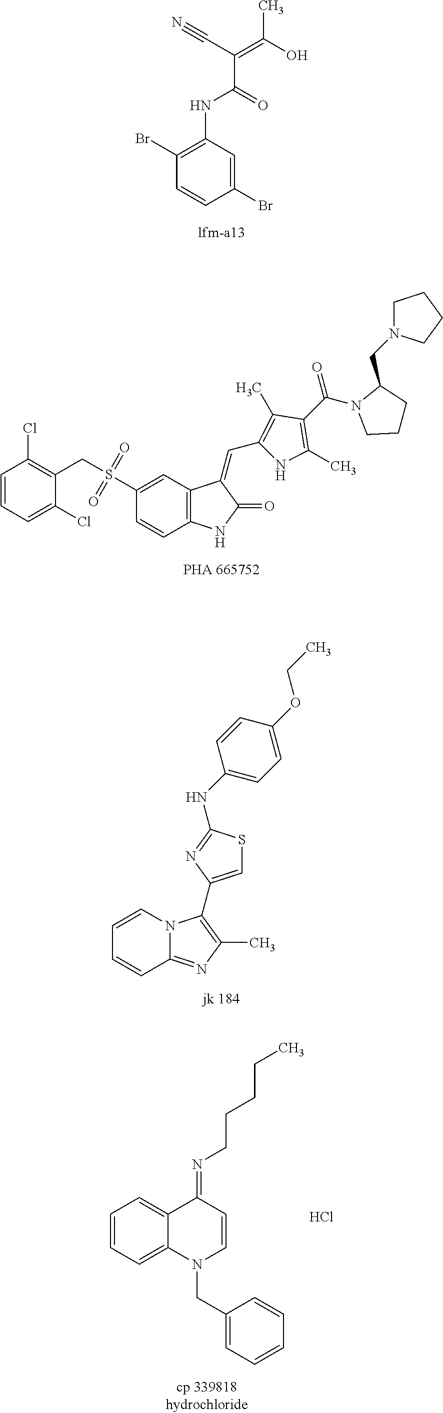 [SMILES: C/C(O)=C(\C#N)C(=O)Nc1cc(Br)ccc1Br ; Cc1[nH]c(/C=C2\C(=O)Nc3ccc(S(=O)(=O)Cc4c(Cl)cccc4Cl)cc32)c(C)c1C(=O)N1CCC[C@@H]1CN1CCCC1 ; CCOc1ccc(Nc2nc(-c3c(C)nc4ccccn34)cs2)cc1 ; CCCCC/N=c1\ccn(Cc2ccccc2)c2ccccc12 ; Cl]

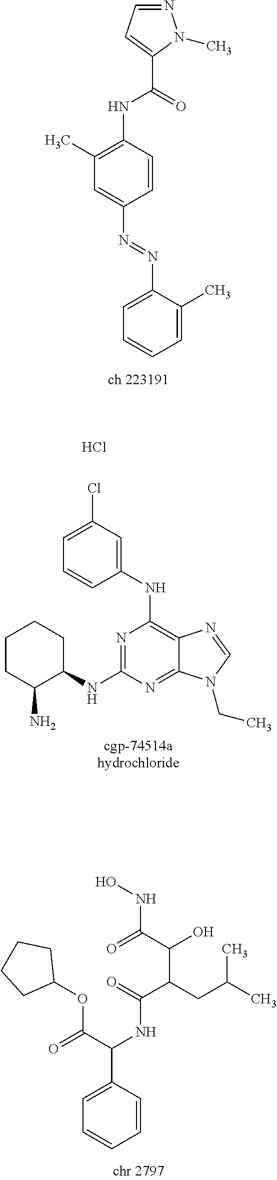

or a pharmaceutically acceptable salt thereof.

In one embodiment, the compounds disclosed herein are further administered in combination with an additional therapeutic agent. In one embodiment, the additional therapeutic agent is selected from Alzheimer's disease medications such as memantine, donepezil (Aricept®), galantamine (Reminyl®), tacrine hydrochloride (Cognex®), and rivastigmine tartrate (Exelon®).

In another aspect, disclosed herein is a method for inhibiting tau phosphorylation comprising administering to a subject a compound selected from sb 206553 hydrochloride, sb 408124, nnc 55-0396 dihydrochloride, win 64338 hydrochloride, u-75302, rs 17053 hydrochloride, lfm-a13, PHA 665752, jk 184, cp 339818 hydrochloride, ch 223191, cgp-74514a hydrochloride, or chr 2797.

In a further aspect, disclosed herein is a method for inhibiting tau phosphorylation in a cell comprising introducing to the cell a compound selected from sb 206553 hydrochloride, sb 408124, nnc 55-0396 dihydrochloride, win 64338 hydrochloride, u-75302, rs 17053 hydrochloride, lfm-a13, PHA 665752, jk 184, cp 339818 hydrochloride, ch 223191, cgp-74514a hydrochloride, or chr 2797. In some embodiments, the cell is a mammalian cell. In some embodiments, the cell is a human cell.

As further disclosed herein, the systematic Alzheimer's disease drug repositioning (SMART) framework integrates experimental and computational biology methods systematically with public transcriptomic profile data to enable fast-track identification and confirmation of novel drug candidates for AD therapy. Using this systematic Alzheimer's disease drug repositioning (SMART) framework, additional compounds were identified that regulate levels of tau phosphorylation and are useful for treating or preventing Alzheimer's disease.

In one aspect, disclosed herein is a method for treating or preventing Alzheimer's disease comprising administering to a subject in need thereof a therapeutically effective amount of a compound selected from the following:

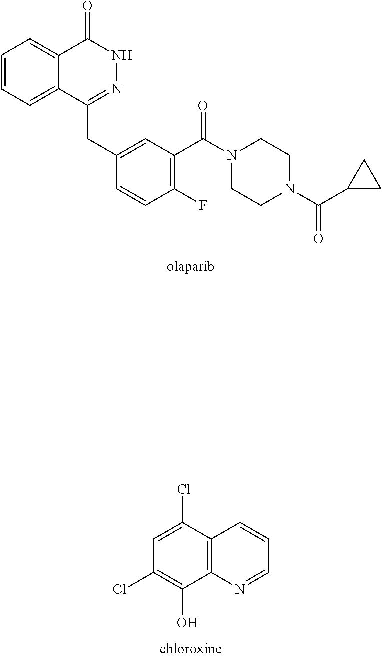 [SMILES: O=C(c1cc(Cc2n[nH]c(=O)c3ccccc23)ccc1F)N1CCN(C(=O)C2CC2)CC1 ; Oc1c(Cl)cc(Cl)c2cccnc12]

or a pharmaceutically acceptable salt thereof.

In one embodiment, the compound is olaparib. In one embodiment, the compound is chloroxine.

In another aspect, disclosed herein is a method for inhibiting tau phosphorylation comprising administering to a subject a compound selected from olaparib or chloroxine.

In a further aspect, disclosed herein is a method for inhibiting tau phosphorylation in a cell comprising introducing to the cell a compound selected from olaparib or chloroxine.

In one aspect, disclosed herein is a method for treating or preventing Alzheimer's disease comprising administering to a subject in need thereof a therapeutically effective amount of a compound selected from the following:

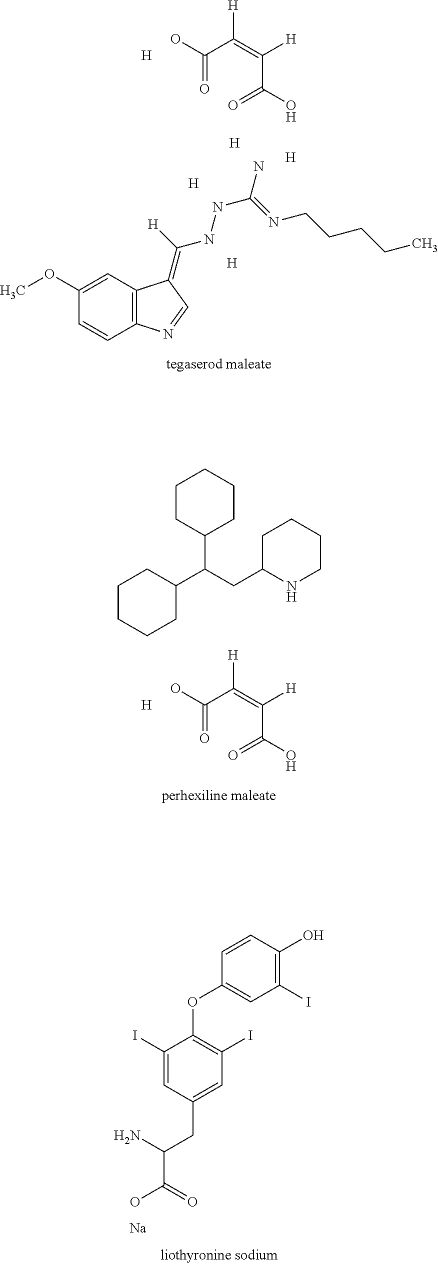

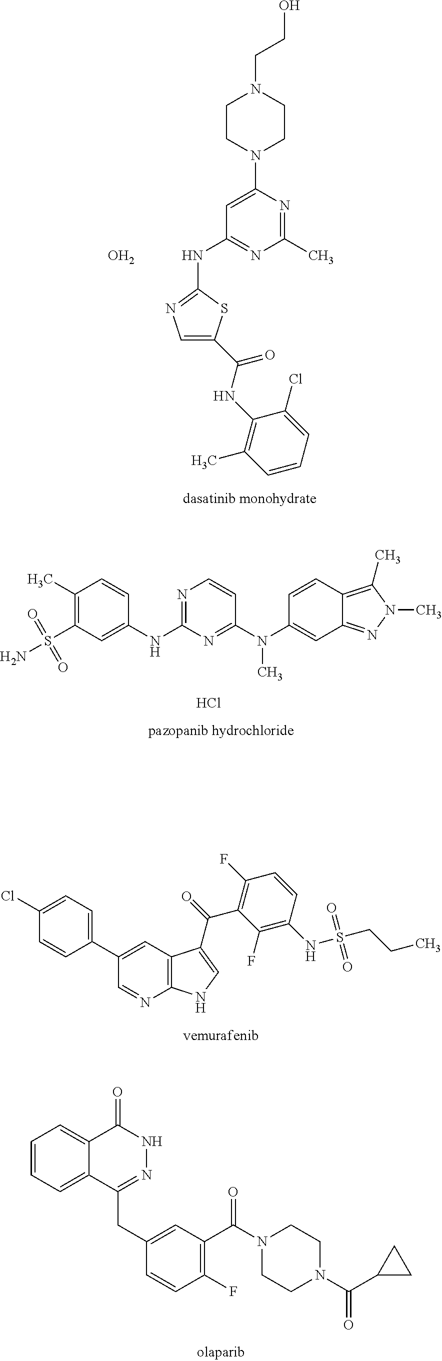 [SMILES: O ; Cc1nc(Nc2ncc(C(=O)Nc3c(C)cccc3Cl)s2)cc(N2CCN(CCO)CC2)n1 ; Cc1ccc(Nc2nccc(N(C)c3ccc4c(C)n(C)nc4c3)n2)cc1S(N)(=O)=O ; Cl ; CCCS(=O)(=O)Nc1ccc(F)c(C(=O)c2c[nH]c3ncc(-c4ccc(Cl)cc4)cc23)c1F ; O=C(c1cc(Cc2n[nH]c(=O)c3ccccc23)ccc1F)N1CCN(C(=O)C2CC2)CC1]

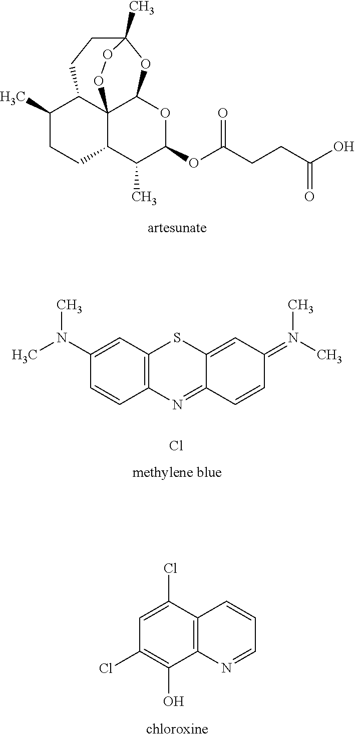

or a pharmaceutically acceptable salt thereof.

In another aspect, disclosed herein is a method for inhibiting tau phosphorylation comprising administering to a subject a compound selected from tegaserod maleate, perhexiline maleate, liothyronine sodium, dasatinib monohydrate, pazopanib hydrochloride, vemurafenib, olaparib, artesunate, methylene blue, or chloroxine; or in some embodiments a drug analog such as alosetron, Levothyroxine, Imatinib, Nilotinib, Bosutinib, Ponatinib, Bafetinib, Dabrafenib, Niraparib, Talazoparib, Artester, Arteether, Deoxyarteether, Artemether, Artemisinin, Dihydroartemisinin, Artelinic acid, Artemotil, Arterolane, Chloroquine, Primaquine, or Pentaquine.

In a further aspect, disclosed herein is a method for inhibiting tau phosphorylation in a cell comprising introducing to the cell a compound selected from tegaserod maleate, perhexiline maleate, liothyronine sodium, dasatinib monohydrate, pazopanib hydrochloride, vemurafenib, olaparib, artesunate, methylene blue, or chloroxine; or in some embodiments a drug analog such as alosetron, Levothyroxine, Imatinib, Nilotinib, Bosutinib, Ponatinib, Bafetinib, Dabrafenib, Niraparib, Talazoparib, Artester, Arteether, Deoxyarteether, Artemether, Artemisinin, Dihydroartemisinin, Artelinic acid, Artemotil, Arterolane, Chloroquine, Primaquine, or Pentaquine.

### BRIEF DESCRIPTION OF THE DRAWINGS

The accompanying figures, which are incorporated in and constitute a part of this specification, illustrate several aspects described below.

FIG. 1. High-content screening. FAD-transfected neural stem cells were 3D cultured in matrigel and treated with each compound. Cells were stained with phospho-tau antibody (AT8). Images covering the whole well were taken and quantified as the readout.

FIG. 2. Image processing workflow of the SMART framework. (A) Schematic of the image processing workflow of the SMART framework. (B) An example result from the image processing is shown.

FIG. 3. Whole-well images showing that treatment with primary hit compounds reduces p-tau (phosphorylated tau) staining (black).

FIG. 4. The workflow of the SMART platform workflow. 17 primary hits were selected to make 60 predictions (box labelled “New Hit Candidates”). Four were validated (box labelled “Validate New Hits”) as highly effective in inhibiting pTau (phospho-Tau or phosphorylated Tau).

FIG. 5. Pilot SMART screen used 20 primary hits to predict 5 new compound hits that inhibit pTau. Validated using the AD-in-a-dish model.

FIG. 6. Graph theory analysis showing relationships among target signatures, predicted hit candidates, and validated hits. (Left) 17 primary hits (blue) predicted 85 candidate compounds. Five (yellow) almost completely inhibit pTau in validation studies while another 5 (green) partially inhibit pTau; (Right) degree-sorted version of the connected sub-graph in (A) reveals that 4 of 5 yellow nodes have a degree larger than 4, which ranked among top 18 of all 85 predicted compounds in degree of a node.

FIG. 7. Ivermectin and its 16 predictions, which include 4 out of 5 yellow nodes confirmed by cell based validations.

FIG. 8. MG624 was added to cells at week 1, 2, 3, 4, and 5. Cells were fixed and stained at week 6. MG624 significantly reduced tau phosphorylation even when added after week 4, when tau phosphorylation should already be fully developed.

FIG. 9. The development of phosphorylated tau in 3D culture neural stem cell model. Neurites expressing phosphorylated tau start to appear at week 2. Tau phosphorylation reaches its maximum at week 4 and is sustained after that.

FIG. 10. The structure for the proposed deep belief network implemented in the SMART framework for Alzheimer's drug repositioning.

FIG. 11. Generating single-clonal cell lines by FACS-assisted 96-well cloning. a. Fluorescence images of single-cell-derived colonies. b. Western blot analysis of Aβ in conditioned media collected from single clonal ReN cells. c. Fluorescence images of ReN-mGAP before and after single cell cloning. Red, mCherry; Green, GFP. d. Aβ40 and 42 levels in media from single clonal ReN cells.

FIG. 12. Confocal immunofluorescence of β-amyloid and p-tau in single-clonal FAD and control ReN cell lines. Cells were 3D-differentiated (thin-layer format) for 7 weeks. (Left panel) β-amyloid plaque (blue). Neuronal cells were co-stained with anti-MAP2 (red*). (Right panel) Immunofluorescence of p-tau levels using anti-PHF1.

FIG. 13. Detection of Sarkosyl-insoluble fibril structures in 14-week-differentiated AD ReN cells in 3D culture (ReN-mAP). Electron microscopy shows differential forms of fibril structures. Small arrowheads, helical twist of the fibril structures.

FIG. 14. Spontaneous firing in 3D-differentiated control (ReN-m # D3) and AD ReN (ReN-mAP # D1) cells by Ca²⁺ imaging. a. Time-lapse images (˜4 sec) over 6 min. Arrowheads indicate cell body and neurites with spontaneous firing. b. Graphs showing Ca²⁺ changes in cells with arrows in a. c. Elevated GCaMP6/Ca²⁺ in neurites and cell bodies from 7-week 3D-differentiated AD ReN cells. Arrowheads, abnormal cell bodies and neurites with high Ca²⁺.

FIG. 15. RNA-seq and canonical pathway analysis shows significant overlaps between clonal 3D AD models and human AD patient brains. a. Pearson correlations of global gene expression profile among 2D undifferentiated control ReN cells, 3D control (G2# B2on), AD # A5 (# A5, moderate Aβ42/40 ratio ˜0.2), AD # D4 (# D4, high Aβ42/40 ratio, ˜1.4), and AD # H10 (# H10, extra high Aβ42/40 ratio, ˜1.7). Units are log CPM. b. Volcano plots show −log₁₀(FDR) vs log FC distribution for G2# B2 (control) vs AD # A5 (AD), AD # A5 DMSO vs AD # A5 BSI (BACE inhibitor, Ly2886721), and AD # A5 DMSO vs AD # A5 GSM (gamma secretase modulator, SGSM15606) transcriptomic signatures. Significantly differentially expressed genes in blue=log FC<−1.0, FDR<0.05|red=log FC>1.0, FDR<0.05. c. Canonical pathway analysis between G2# B2 and AD # A5 (Ingenuity pathway analysis, Qiagen). d. Analysis of common canonical pathways. The pathway analysis among G2# B2 vs AD # A5, AD # A5 DMSO vs AD # A5 BSI, and AD # A5 DMSO vs AD # A5 GSM. Activation z-scores indicate that majority of decreased pathways in AD # A5 are restored by BSI and/or GSM treatments. e. Comparison of enriched pathways between the 3D G2# B2 vs AD # A5 and normal brains vs AD patient brains (from the publicly available datasets). The analysis showed many common pathways significantly decreased both in human AD brains and the 3D AD # A5 samples.

FIG. 16. Validating the impact of primary hit candidates using multiple human AD cell lines with different Aβ42/40 ratios. Control and AD cells were differentiated for 6 weeks in 3D culture conditions with drug treatments in last 3 weeks. Levels of insoluble p-tau (pThr181tau) and total tau were measured by Mesoscale ELISA while actin and Tuj1 (neural marker) were measured by quantitative dot blot analyses with LiCor infrared laser system. p-Tau levels were normalized either by Tuj1 or total tau. Relative decreases of phospho tau levels in each experiment (n=4 to 5) were color-coded and scored.

FIG. 17. Validation of primary hit candidates. Primary hit candidates were confirmed using Western blot analysis (a) and quantitative immunofluorescence staining in 3D AD models with high Aβ42/40 ratios (# HReN and # A4H1) (b). PHF1 pSer396/Ser404 tau antibody was used to detect changes in phospho tau in 3D AD # HReN cells treated with DMSO vehicle, ebselen, or leflunomide.

FIG. 18A-B. Systematic modeling of RNAseq data reveals shared changes for two screening hits. (a) PPI networks involving APP, MAPT as well as 15 down-regulated (dark grey: IFNA1, IFNA2, TLR7, IRF3, IFNAR1, TLR9, IL1B, IFNG, TNF, TGM2, MAP3K7, ZAP70, EIF2AK2, IL29, PRL) and 7 up-regulated (light grey: SOCS1, EGF, IFIH1, IL1RN, BTK, GAPDH, MAPK1) genes after separate treatments of ebselen or leflunomide. Red edges illustrate PPI connecting APP to members of a group of 7 significantly changed genes. PPI information was extracted from STRING database version 10.5 with the cutoff for confidence score at 0.4. (b) A sub-network involving 12 genes and 6 pathways are significantly down-regulated (dark grey nodes with log FC<−1.5) by the treatments of candidates ebselen and leflunomide.

### DETAILED DESCRIPTION

Disclosed herein are compounds and methods for the treatment and/or prevention of Alzheimer's disease. To develop a high-throughput in vitro cellular model, a modified neural stem cell model was used which gradually develops tau pathology during culture. Using the modified high-throughput in vitro cellular model, several compounds were identified that regulate levels of tau phosphorylation and are useful for treating or preventing Alzheimer's disease.

As further disclosed herein, the systematic Alzheimer's disease drug repositioning (SMART) framework integrates experimental and computational biology methods systematically with public transcriptomic profile data to enable fast-track identification and confirmation of novel drug candidates for AD therapy. Using this systematic Alzheimer's disease drug repositioning (SMART) framework, additional compounds were identified that regulate levels of tau phosphorylation and are useful for treating or preventing Alzheimer's disease.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this invention belongs. The following definitions are provided for the full understanding of terms used in this specification.

### Terminology

As used in the specification and claims, the singular form “a,” “an,” and “the” include plural references unless the context clearly dictates otherwise. For example, the term “a cell” includes a plurality of cells, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject.

The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

As used herein, the term “mixture” can include solutions in which the components of the mixture are completely miscible, as well as suspensions and emulsions, in which the components of the mixture are not completely miscible.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human. In some embodiments, the pharmacokinetic profiles of the systems of the present invention are similar for male and female subjects.

As used herein, the term “controlled-release” or “controlled-release drug delivery” or “extended release” refers to release or administration of a drug from a given dosage form in a controlled fashion in order to achieve the desired pharmacokinetic profile in vivo. An aspect of “controlled” drug delivery is the ability to manipulate the formulation and/or dosage form in order to establish the desired kinetics of drug release.

The phrases “concurrent administration”, “administration in combination”, “simultaneous administration” or “administered simultaneously” as used herein, means that the compounds are administered at the same point in time or immediately following one another. In the latter case, the two compounds are administered at times sufficiently close that the results observed are indistinguishable from those achieved when the compounds are administered at the same point in time.

### Methods of Treatment—Alzheimer's Disease

Disclosed herein are compounds and methods for the treatment and/or prevention of Alzheimer's disease. To develop a high-throughput in vitro cellular model, a modified neural stem cell model was used which gradually develops tau pathology during culture. Using the modified high-throughput in vitro cellular model, several compounds were identified that regulate levels of tau phosphorylation and are useful for treating or preventing Alzheimer's disease.

In one aspect, disclosed herein is a method for treating or preventing Alzheimer's disease comprising administering to a subject in need thereof a therapeutically effective amount of a compound selected from the following compounds listed in Table 1:

TABLE 1
Compounds for Treating or Preventing Alzheimer's Disease

| Drug/Compound | Structure |
|---|---|
| sb 206553 hydrochloride | 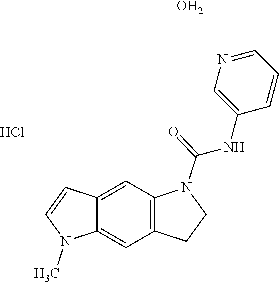 [SMILES: O ; Cl ; Cn1ccc2cc3c(cc21)CCN3C(=O)Nc1cccnc1] |
| sb 408124 | 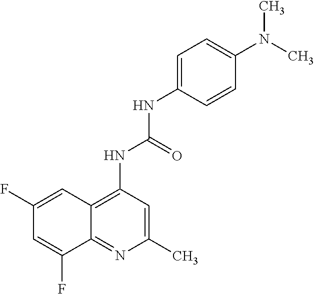 [SMILES: Cc1cc(NC(=O)Nc2ccc(N(C)C)cc2)c2cc(F)cc(F)c2n1] |
| nnc 55-0396 dihydrochloride | 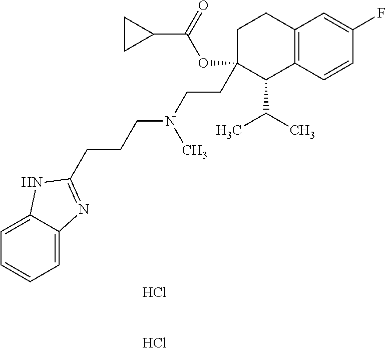 [SMILES: CC(C)[C@H]1c2ccc(F)cc2CC[C@@]1(CCN(C)CCCc1nc2ccccc2[nH]1)OC(=O)C1CC1 ; Cl ; Cl] |
| win 64338 hydrochloride | 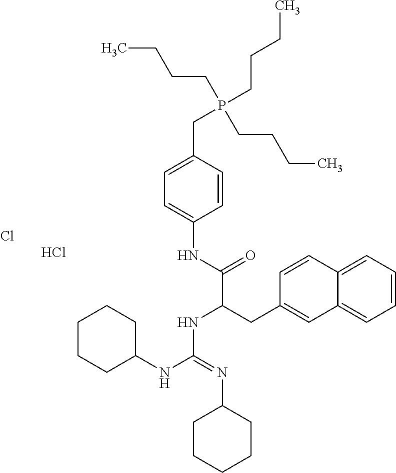 |
| u-75302 | 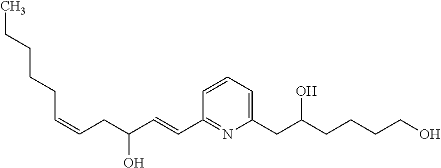 [SMILES: CCCCC/C=C\CC(O)/C=C/c1cccc(CC(O)CCCCO)n1] |
| rs 17053 hydrochloride | 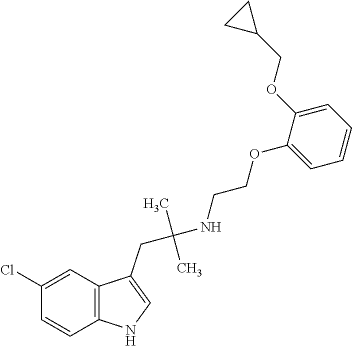 [SMILES: CC(C)(Cc1c[nH]c2ccc(Cl)cc12)NCCOc1ccccc1OCC1CC1] |
| lfm-a13 | 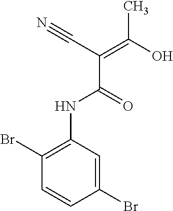 [SMILES: C/C(O)=C(\C#N)C(=O)Nc1cc(Br)ccc1Br] |
| PHA 665752 | 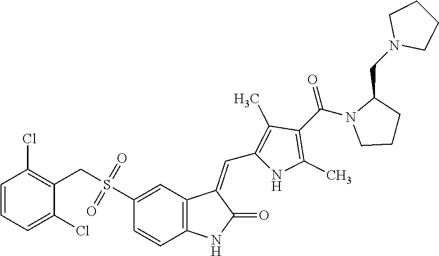 [SMILES: Cc1[nH]c(/C=C2\C(=O)Nc3ccc(S(=O)(=O)Cc4c(Cl)cccc4Cl)cc32)c(C)c1C(=O)N1CCC[C@@H]1CN1CCCC1] |
| jk 184 | 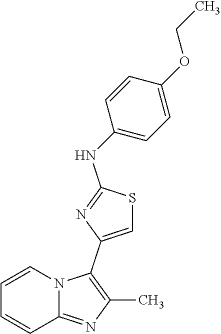 [SMILES: CCOc1ccc(Nc2nc(-c3c(C)nc4ccccn34)cs2)cc1] |
| cp 339818 hydrochloride | 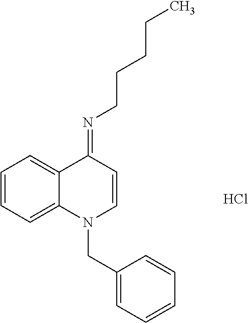 [SMILES: CCCCC/N=c1\ccn(Cc2ccccc2)c2ccccc12 ; Cl] |
| ch 223191 | 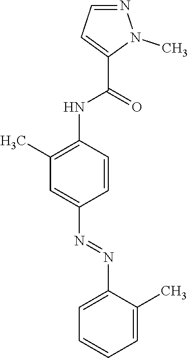 |
| cgp-74514a hydrochloride | 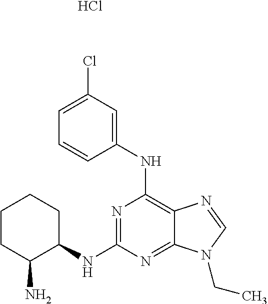 [SMILES: Cl ; CCn1cnc2c(Nc3cccc(Cl)c3)nc(N[C@@H]3CCCC[C@@H]3N)nc21] |
| chr 2797 | 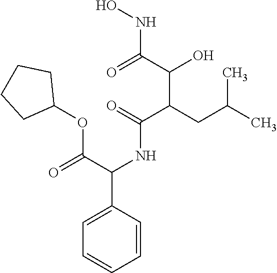 [SMILES: CC(C)CC(C(=O)NC(C(=O)OC1CCCC1)c1ccccc1)C(O)C(=O)NO] |

or a pharmaceutically acceptable salt thereof.

In one embodiment, the compound is sb 206553 hydrochloride. In one embodiment, the compound is sb 408124. In one embodiment, the compound is nnc 55-0396 dihydrochloride. In one embodiment, the compound is win 64338 hydrochloride. In one embodiment, the compound is u-75302. In one embodiment, the compound is rs 17053 hydrochloride. In one embodiment, the compound is lfm-a13. In one embodiment, the compound is PHA 665752. In one embodiment, the compound is jk 184. In one embodiment, the compound is cp 339818 hydrochloride. In one embodiment, the compound is ch 223191. In one embodiment, the compound is cgp-74514a hydrochloride. In one embodiment, the compound is or chr 2797.

In another aspect, disclosed herein is a method for inhibiting tau phosphorylation comprising administering to a subject a compound selected from sb 206553 hydrochloride, sb 408124, nnc 55-0396 dihydrochloride, win 64338 hydrochloride, u-75302, rs 17053 hydrochloride, lfm-a13, PHA 665752, jk 184, cp 339818 hydrochloride, ch 223191, cgp-74514a hydrochloride, or chr 2797.

In a further aspect, disclosed herein is a method for inhibiting tau phosphorylation in a cell comprising introducing to the cell a compound selected from sb 206553 hydrochloride, sb 408124, nnc 55-0396 dihydrochloride, win 64338 hydrochloride, u-75302, rs 17053 hydrochloride, lfm-a13, PHA 665752, jk 184, cp 339818 hydrochloride, ch 223191, cgp-74514a hydrochloride, or chr 2797.

In some embodiments, the cell is a mammalian cell. In some embodiments, the cell is a human cell.

In one aspect, disclosed herein is a method for treating or preventing Alzheimer's disease comprising administering to a subject in need thereof a therapeutically effective amount of a compound selected from the following compounds listed in Table 2:

TABLE 2
List of 38 AD therapeutic agents identified in a high-throughput 3D cell line screen

| Drug/Compound | Structure |
|---|---|
| sb 206553 hydrochloride | 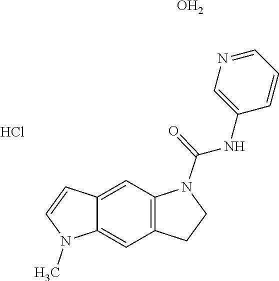 [SMILES: O ; Cl ; Cn1ccc2cc3c(cc21)CCN3C(=O)Nc1cccnc1] |
| rs 67333 hydrochloride | 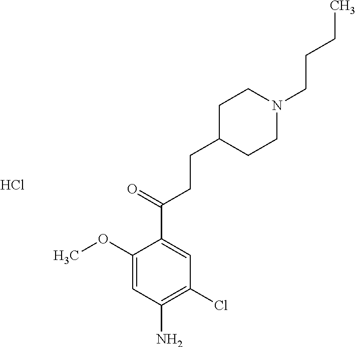 [SMILES: Cl ; CCCCN1CCC(CCC(=O)c2cc(Cl)c(N)cc2OC)CC1] |
| mg 624 | 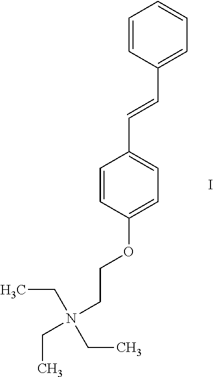 |
| ro 90-7501 | 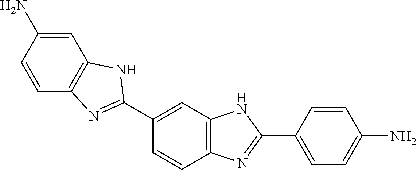 [SMILES: Nc1ccc(-c2nc3ccc(-c4nc5ccc(N)cc5[nH]4)cc3[nH]2)cc1] |
| y 29794 oxalate | 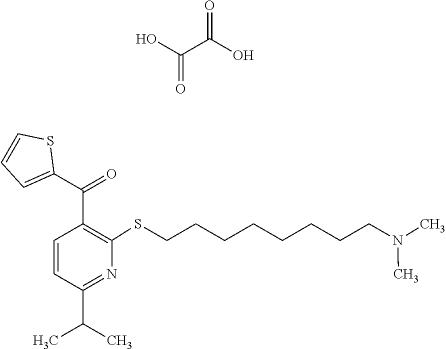 [SMILES: O=C(O)C(=O)O ; CC(C)c1ccc(C(=O)c2cccs2)c(SCCCCCCCCN(C)C)n1] |
| sb 408124 | 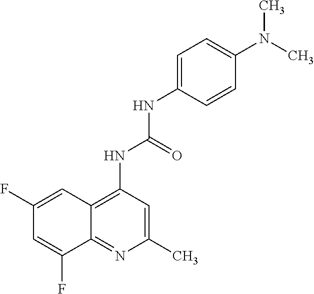 [SMILES: Cc1cc(NC(=O)Nc2ccc(N(C)C)cc2)c2cc(F)cc(F)c2n1] |
| bio | 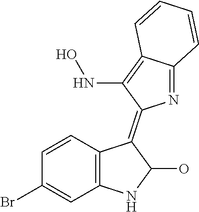 [SMILES: ONC1=c2ccccc2=N/C1=C1/c2ccc(Br)cc2NC1O] |
| cd 1530 | 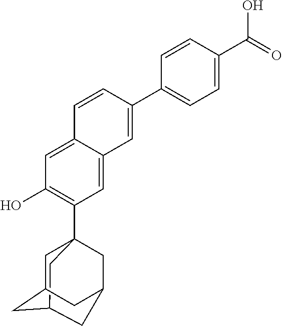 [SMILES: O=C(O)c1ccc(-c2ccc3cc(O)c(C45CC6CC(CC(C6)C4)C5)cc3c2)cc1] |
| ttnpb | 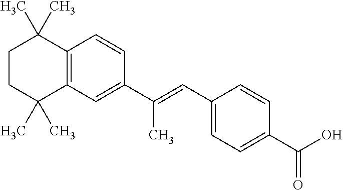 [SMILES: C/C(=C\c1ccc(C(=O)O)cc1)c1ccc2c(c1)C(C)(C)CCC2(C)C] |
| nnc 55-0396 dihydrochloride | 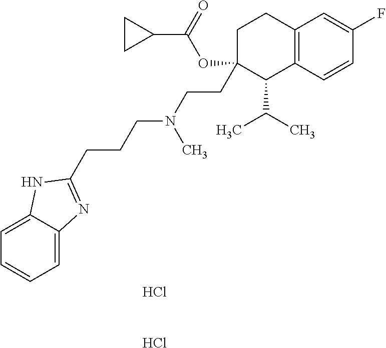 [SMILES: CC(C)[C@H]1c2ccc(F)cc2CC[C@@]1(CCN(C)CCCc1nc2ccccc2[nH]1)OC(=O)C1CC1 ; Cl ; Cl] |
| win 64338 hydrochloride | 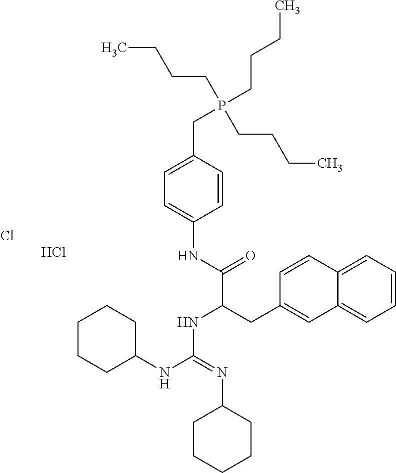 |
| u-75302 | 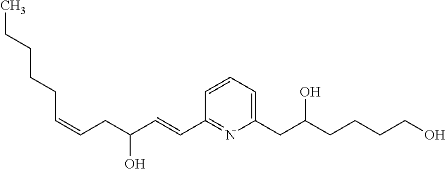 [SMILES: CCCCC/C=C\CC(O)/C=C/c1cccc(CC(O)CCCCO)n1] |
| rs 17053 hydrochloride | 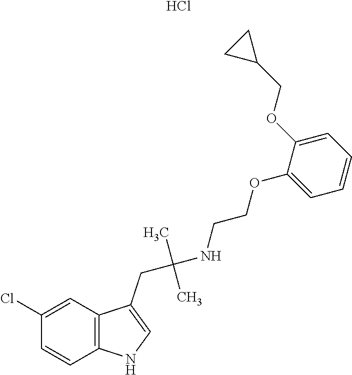 [SMILES: Cl ; CC(C)(Cc1c[nH]c2ccc(Cl)cc12)NCCOc1ccccc1OCC1CC1] |
| rottlerin | 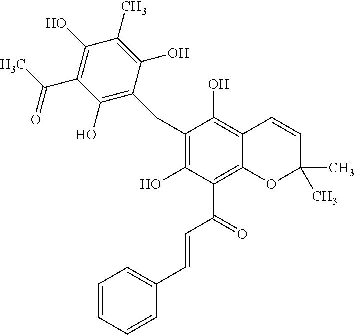 [SMILES: CC(=O)c1c(O)c(C)c(O)c(Cc2c(O)c3c(c(C(=O)/C=C/c4ccccc4)c2O)OC(C)(C)C=C3)c1O] |
| arcyriaflavin a | 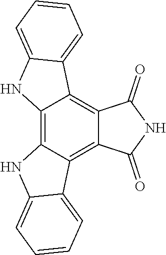 [SMILES: O=C1NC(=O)c2c1c1c3ccccc3[nH]c1c1[nH]c3ccccc3c21] |
| pp1 | 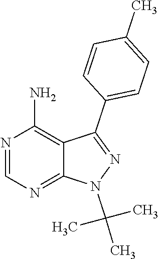 [SMILES: Cc1ccc(-c2nn(C(C)(C)C)c3ncnc(N)c23)cc1] |
| lfm-a13 | 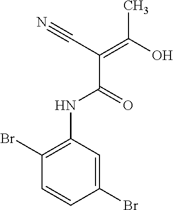 [SMILES: C/C(O)=C(\C#N)C(=O)Nc1cc(Br)ccc1Br] |
| PHA 665752 | 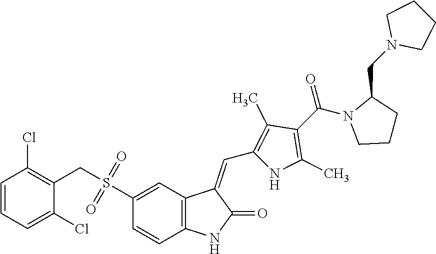 [SMILES: Cc1[nH]c(/C=C2\C(=O)Nc3ccc(S(=O)(=O)Cc4c(Cl)cccc4Cl)cc32)c(C)c1C(=O)N1CCC[C@@H]1CN1CCCC1] |
| jk 184 | 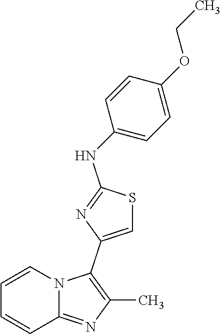 [SMILES: CCOc1ccc(Nc2nc(-c3c(C)nc4ccccn34)cs2)cc1] |
| cp 339818 hydrochloride | 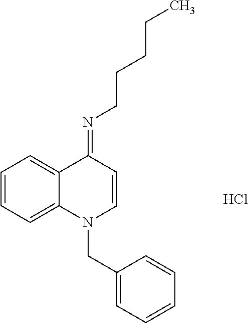 [SMILES: CCCCC/N=c1\ccn(Cc2ccccc2)c2ccccc12 ; Cl] |
| ch 223191 | 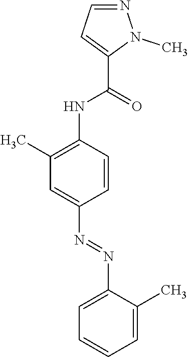 |
| cgp-74514a hydrochloride | 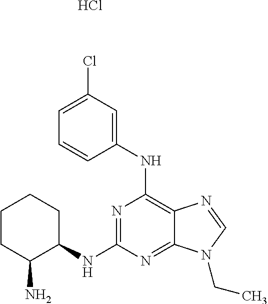 [SMILES: Cl ; CCn1cnc2c(Nc3cccc(Cl)c3)nc(N[C@@H]3CCCC[C@@H]3N)nc21] |
| baicalein | 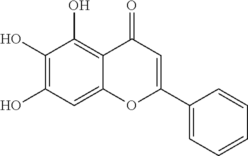 [SMILES: O=c1cc(-c2ccccc2)oc2cc(O)c(O)c(O)c12] |
| actinonin | 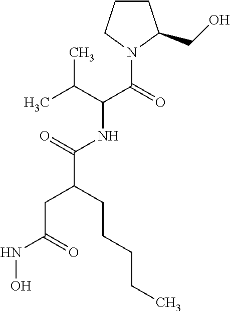 [SMILES: CCCCCC(CC(=O)NO)C(=O)NC(C(=O)N1CCC[C@H]1CO)C(C)C] |
| 1,4-pbit dihydrobromide | 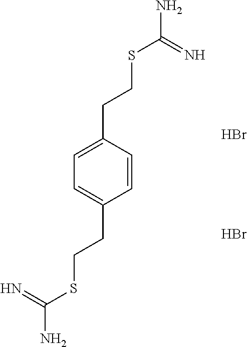 [SMILES: N=C(N)SCCc1ccc(CCSC(=N)N)cc1 ; Br ; Br] |
| chr 2797 | 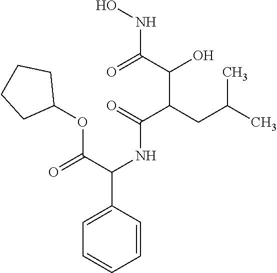 [SMILES: CC(C)CC(C(=O)NC(C(=O)OC1CCCC1)c1ccccc1)C(O)C(=O)NO] |
| ebselen | 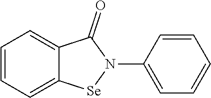 [SMILES: O=c1c2ccccc2[se]n1-c1ccccc1] |
| ivermectin | 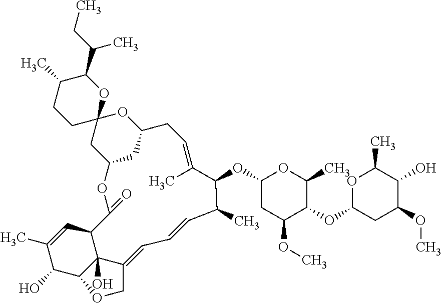 |
| retinoic acid | 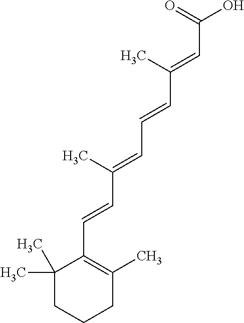 [SMILES: CC1=C(/C=C/C(C)=C/C=C/C(C)=C/C(=O)O)C(C)(C)CCC1] |
| loperamide hydrochloride | 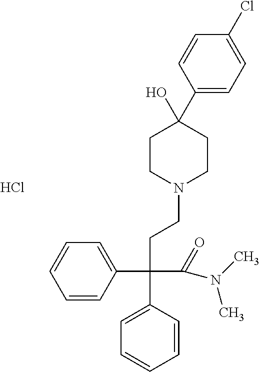 [SMILES: Cl ; CN(C)C(=O)C(CCN1CCC(O)(c2ccc(Cl)cc2)CC1)(c1ccccc1)c1ccccc1] |
| nifedipine | 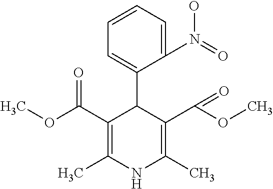 |
| rapamycin/sirolimus | 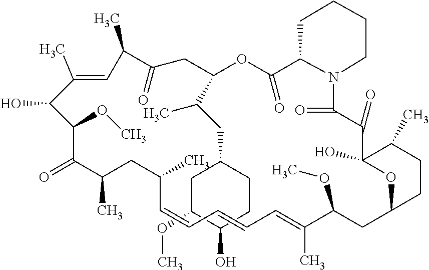 [SMILES: CO[C@H]1C[C@H]2CC[C@@H](C)[C@@](O)(O2)C(=O)C(=O)N2CCCC[C@H]2C(=O)O[C@H](C(C)C[C@@H]2CC[C@@H](O)[C@H](OC)C2)CC(=O)[C@H](C)/C=C(\C)[C@@H](O)[C@@H](OC)C(=O)[C@H](C)C[C@H](C)\C=C/C=C/C=C/1C] |
| fluticasone propionate | 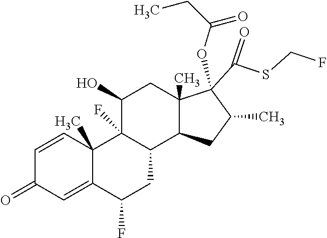 [SMILES: CCC(=O)O[C@]1(C(=O)SCF)[C@H](C)C[C@H]2[C@@H]3C[C@H](F)C4=CC(=O)C=C[C@]4(C)[C@@]3(F)[C@@H](O)C[C@@]21C] |
| cyclosporin a | 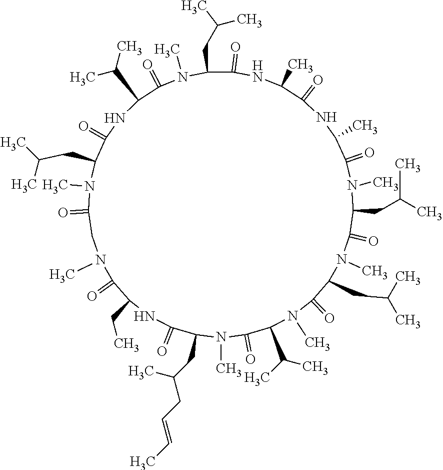 [SMILES: C/C=C/CC(C)C[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C] |
| pentamidine isethionate | 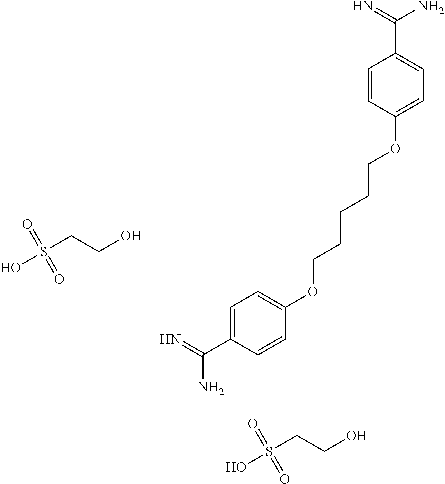 [SMILES: O=S(=O)(O)CCO ; N=C(N)c1ccc(OCCCCCOc2ccc(C(=N)N)cc2)cc1 ; O=S(=O)(O)CCO] |
| leflunomide | 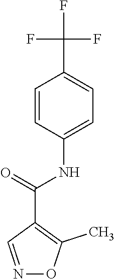 [SMILES: Cc1oncc1C(=O)Nc1ccc(C(F)(F)F)cc1] |
| bromoacetyl alprenolol menthane | 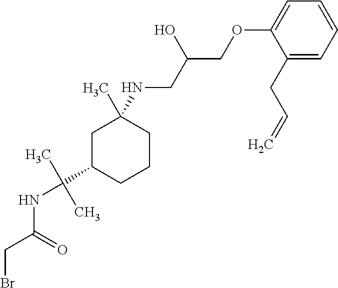 [SMILES: C=CCc1ccccc1OCC(O)CN[C@]1(C)CCC[C@H](C(C)(C)NC(=O)CBr)C1] |
| mibefradil dihydrochloride | 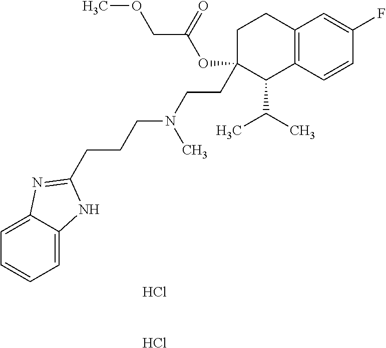 [SMILES: COCC(=O)O[C@]1(CCN(C)CCCc2nc3ccccc3[nH]2)CCc2cc(F)ccc2[C@@H]1C(C)C ; Cl ; Cl] |

or a pharmaceutically acceptable salt thereof.

In another aspect, disclosed herein is a method for inhibiting tau phosphorylation comprising administering to a subject a compound selected from sb 206553 hydrochloride, rs 67333 hydrochloride, mg 624, ro 90-7501, y 29794 oxalate, sb 408124, bio, cd 1530, ttnpb, nnc 55-0396 dihydrochloride, win 64338 hydrochloride, u-75302, rs 17053 hydrochloride, rottlerin, arcyriaflavin a, pp1, lfm-a13, PHA 665752, jk 184, cp 339818 hydrochloride, ch 223191, cgp-74514a hydrochloride, baicalein, actinonin, 1,4-pbit dihydrobromide, chr 2797, ebselen, ivermectin, retinoic acid, loperamide hydrochloride, nifedipine, rapamycin/sirolimus, fluticasone propionate, cyclosporin A, pentamidine isethionate, leflunomide, bromoacetyl alprenolol menthane, mibefradil, or dihydrochloride.

In a further aspect, disclosed herein is a method for inhibiting tau phosphorylation in a cell comprising introducing to the cell a compound selected from sb 206553 hydrochloride, rs 67333 hydrochloride, mg 624, ro 90-7501, y 29794 oxalate, sb 408124, bio, cd 1530, ttnpb, nnc 55-0396 dihydrochloride, win 64338 hydrochloride, u-75302, rs 17053 hydrochloride, rottlerin, arcyriaflavin a, pp1, lfm-a13, PHA 665752, jk 184, cp 339818 hydrochloride, ch 223191, cgp-74514a hydrochloride, baicalein, actinonin, 1,4-pbit dihydrobromide, chr 2797, ebselen, ivermectin, retinoic acid, loperamide hydrochloride, nifedipine, rapamycin/sirolimus, fluticasone propionate, cyclosporin A, pentamidine isethionate, leflunomide, bromoacetyl alprenolol menthane, mibefradil, or dihydrochloride.

In one embodiment, the compound is sb 206553 hydrochloride. In one embodiment, the compound is rs 67333 hydrochloride. In one embodiment, the compound is mg 624. In one embodiment, the compound is ro 90-7501. In one embodiment, the compound is y 29794 oxalate. In one embodiment, the compound is sb 408124. In one embodiment, the compound is bio. In one embodiment, the compound is cd 1530. In one embodiment, the compound is ttnpb. In one embodiment, the compound is nnc 55-0396 dihydrochloride. In one embodiment, the compound is win 64338 hydrochloride. In one embodiment, the compound is u-75302. In one embodiment, the compound is rs 17053 hydrochloride. In one embodiment, the compound is rottlerin. In one embodiment, the compound is arcyriaflavin a. In one embodiment, the compound is pp1. In one embodiment, the compound is lfm-a13. In one embodiment, the compound is PHA 665752. In one embodiment, the compound is jk 184. In one embodiment, the compound is cp 339818 hydrochloride. In one embodiment, the compound is ch 223191. In one embodiment, the compound is cgp-74514a hydrochloride. In one embodiment, the compound is baicalein. In one embodiment, the compound is actinonin. In one embodiment, the compound is 1,4-pbit dihydrobromide. In one embodiment, the compound is chr 2797. In one embodiment, the compound is ebselen. In one embodiment, the compound is ivermectin. In one embodiment, the compound is retinoic acid. In one embodiment, the compound is loperamide hydrochloride. In one embodiment, the compound is nifedipine. In one embodiment, the compound is rapamycin/sirolimus. In one embodiment, the compound is fluticasone propionate. In one embodiment, the compound is cyclosporin A. In one embodiment, the compound is pentamidine isethionate. In one embodiment, the compound is leflunomide. In one embodiment, the compound is bromoacetyl alprenolol menthane. In one embodiment, the compound is mibefradil. In one embodiment, the compound is or dihydrochloride.

As further disclosed herein, the systematic Alzheimer's disease drug repositioning (SMART) framework integrates experimental and computational biology methods systematically with public transcriptomic profile data to enable fast-track identification and confirmation of novel drug candidates for AD therapy. Using this systematic Alzheimer's disease drug repositioning (SMART) framework, additional compounds were identified that regulate levels of tau phosphorylation and are useful for treating or preventing Alzheimer's disease.

In one aspect, disclosed herein is a method for treating or preventing Alzheimer's disease comprising administering to a subject in need thereof a therapeutically effective amount of a compound selected from the following compounds listed in Table 3:

TABLE 3
Compounds for Treating or Preventing Alzheimer's Disease

| Drug/Compound | Structure |
|---|---|
| olaparib | 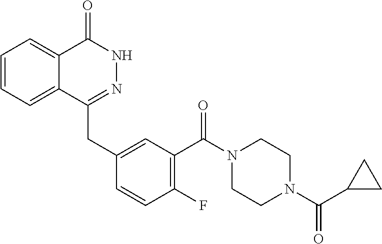 [SMILES: O=C(c1cc(Cc2n[nH]c(=O)c3ccccc23)ccc1F)N1CCN(C(=O)C2CC2)CC1] |
| chloroxine | 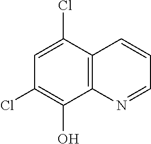 [SMILES: Oc1c(Cl)cc(Cl)c2cccnc12] |

or a pharmaceutically acceptable salt thereof.

In one embodiment, the compound is olaparib. In one embodiment, the compound is chloroxine.

In another aspect, disclosed herein is a method for inhibiting tau phosphorylation comprising administering to a subject a compound selected from olaparib or chloroxine.

In a further aspect, disclosed herein is a method for inhibiting tau phosphorylation in a cell comprising introducing to the cell a compound selected from olaparib or chloroxine. In one aspect, disclosed herein is a method for treating or preventing Alzheimer's disease comprising administering to a subject in need thereof a therapeutically effective amount of a compound selected from the following compounds listed in Table 4:

TABLE 4
List of 10 AD therapeutic agents identified using the SMART platform

| Drug/Compound | Structure |
|---|---|
| tegaserod maleate | 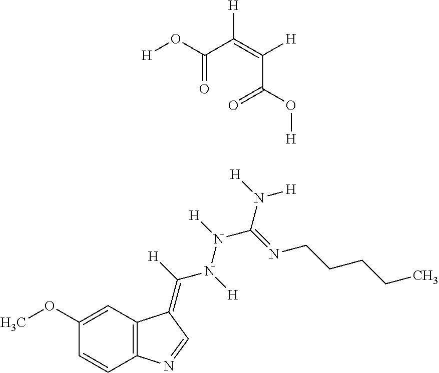 [SMILES: O=C(O)/C=C\C(=O)O ; CCCCC/N=C(\N)NN/C=C1\C=Nc2ccc(OC)cc21] |
| perhexiline maleate | 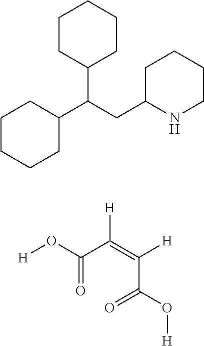 [SMILES: C1CCC(C(CC2CCCCN2)C2CCCCC2)CC1 ; O=C(O)/C=C\C(=O)O] |
| liothyronine sodium | 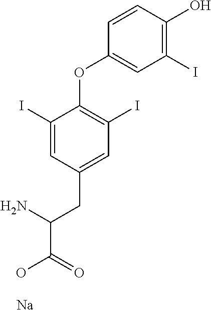 |
| dasatinib monohydrate | 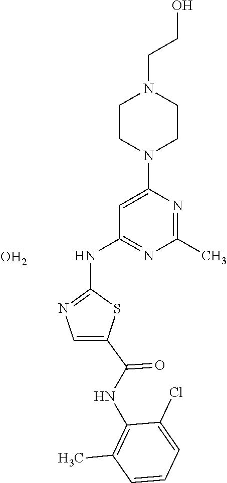 [SMILES: O ; Cc1nc(Nc2ncc(C(=O)Nc3c(C)cccc3Cl)s2)cc(N2CCN(CCO)CC2)n1] |
| pazopanib hydrochloride | 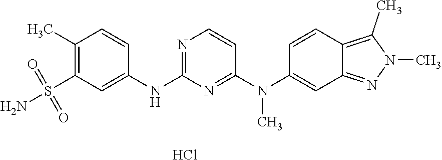 [SMILES: Cc1ccc(Nc2nccc(N(C)c3ccc4c(C)n(C)nc4c3)n2)cc1S(N)(=O)=O ; Cl] |
| vemurafenib | 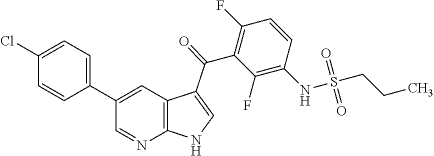 [SMILES: CCCS(=O)(=O)Nc1ccc(F)c(C(=O)c2c[nH]c3ncc(-c4ccc(Cl)cc4)cc23)c1F] |
| olaparib | 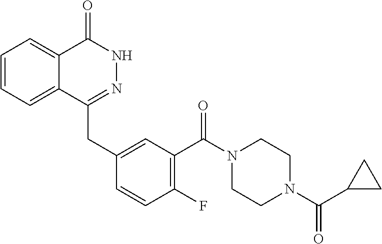 [SMILES: O=C(c1cc(Cc2n[nH]c(=O)c3ccccc23)ccc1F)N1CCN(C(=O)C2CC2)CC1] |
| artesunate | 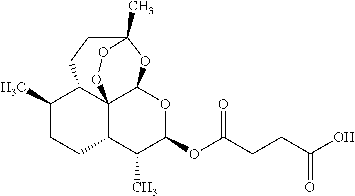 [SMILES: C[C@H]1[C@H](OC(=O)CCC(=O)O)O[C@@H]2O[C@@]3(C)CC[C@H]4[C@H](C)CC[C@@H]1[C@@]24OO3] |
| methylene blue | 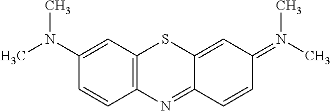 |
| chloroxine | 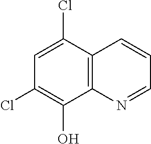 [SMILES: Oc1c(Cl)cc(Cl)c2cccnc12] |

or a pharmaceutically acceptable salt thereof.

In one aspect, disclosed herein is a method for treating or preventing Alzheimer's disease comprising administering to a subject a compound selected from tegaserod maleate, perhexiline maleate, liothyronine sodium, dasatinib monohydrate, pazopanib hydrochloride, vemurafenib, olaparib, artesunate, methylene blue, or chloroxine; or in some embodiments a drug analog such as alosetron, Levothyroxine, Imatinib, Nilotinib, Bosutinib, Ponatinib, Bafetinib, Dabrafenib, Niraparib, Talazoparib, Artester, Arteether, Deoxyarteether, Artemether, Artemisinin, Dihydroartemisinin, Artelinic acid, Artemotil, Arterolane, Chloroquine, Primaquine, or Pentaquine.

In another aspect, disclosed herein is a method for inhibiting tau phosphorylation comprising administering to a subject a compound selected from tegaserod maleate, perhexiline maleate, liothyronine sodium, dasatinib monohydrate, pazopanib hydrochloride, vemurafenib, olaparib, artesunate, methylene blue, or chloroxine; or in some embodiments a drug analog such as alosetron, Levothyroxine, Imatinib, Nilotinib, Bosutinib, Ponatinib, Bafetinib, Dabrafenib, Niraparib, Talazoparib, Artester, Arteether, Deoxyarteether, Artemether, Artemisinin, Dihydroartemisinin, Artelinic acid, Artemotil, Arterolane, Chloroquine, Primaquine, or Pentaquine.

In a further aspect, disclosed herein is a method for inhibiting tau phosphorylation in a cell comprising introducing to the cell a compound selected from tegaserod maleate, perhexiline maleate, liothyronine sodium, dasatinib monohydrate, pazopanib hydrochloride, vemurafenib, olaparib, artesunate, methylene blue, or chloroxine; or in some embodiments a drug analog such as alosetron, Levothyroxine, Imatinib, Nilotinib, Bosutinib, Ponatinib, Bafetinib, Dabrafenib, Niraparib, Talazoparib, Artester, Arteether, Deoxyarteether, Artemether, Artemisinin, Dihydroartemisinin, Artelinic acid, Artemotil, Arterolane, Chloroquine, Primaquine, or Pentaquine.

In one embodiment, the compounds disclosed herein are further administered in combination with an additional therapeutic agent. In one embodiment, the additional therapeutic agent is selected from Alzheimer's disease medications such as memantine, donepezil (Aricept®), galantamine (Reminyl®), tacrine hydrochloride (Cognex®), and rivastigmine tartrate (Exelon®).

In one embodiment, the compound is tegaserod maleate. In one embodiment, the compound is perhexiline maleate. In one embodiment, the compound is liothyronine sodium. In one embodiment, the compound is dasatinib monohydrate. In one embodiment, the compound is pazopanib hydrochloride. In one embodiment, the compound is vemurafenib. In one embodiment, the compound is olaparib. In one embodiment, the compound is artesunate. In one embodiment, the compound is methylene blue. In one embodiment, the compound is chloroxine.

In some embodiments, the cell is a mammalian cell. In some embodiments, the cell is a human cell.

### Combination Therapies—Alzheimer's Disease

In some embodiments, the compounds or compositions described herein can be combined with an additional therapeutic agent. In some embodiments, the additional therapeutic agent is selected from Alzheimer's disease medications such as memantine, donepezil (Aricept®), galantamine (Reminyl®), tacrine hydrochloride (Cognex®), and rivastigmine tartrate (Exelon®).

Donepezil([(R,S)-1-benzyl-4-[(5,6-dimethoxy-1-indanon)-2-yl]-methylpiperidine hydrochloride], also known as Aricept®) is a reversible, noncompetitive, piperidine-type acetylcholinesterase inhibitor. Studies have shown that daily administration of donepezil (5 and 10 mg/day) can lead to significantly improved cognition and global clinical function compared with placebo in short and long-term trials. Donepezil is described, for example, in U.S. Pat. Nos. 6,372,760; 6,245,911; 6,140,321; 5,985,864; and 4,895,841, all of which are incorporated herein by reference in their entireties. Memantine (1-amino-3,5-dimethyl adamantane) is described, for example, in U.S. Pat. Nos. 4,122,193; 4,273,774; 5,061,703, all of which are incorporated herein by reference in their entireties. Memantine is an Alzheimer's disease medication acting on the glutamatergic system by blocking NMDA glutamate receptors. Memantine is advantageous because it lacks the side effects of other NMDA receptor antagonists at similar therapeutic doses.

In some embodiments, the compounds disclosed herein can be combined with experimental drugs targeting different end points of Alzheimer's Disease (AD), such as those of inflammation (microglia), astrocytes, or metabolic (mitochondria).

### Compositions

Compositions, as described herein, comprising an active compound and an excipient of some sort may be useful in a variety of applications.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005). The pharmaceutically acceptable excipients may also include one or more of fillers, binders, lubricants, glidants, disintegrants, and the like.

Exemplary excipients include, but are not limited to, any non-toxic, inert solid, semi-solid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfate, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacilic acid/acrylamide copolymer, polyalkylene oxides such as polyethylene oxide, polypropylene oxide, poly(ethylene oxide-propylene oxide), and a Pluronic polymer, polyoxyethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly(meth)acrylic acid, and esters amide and hydroxyalkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

### EXAMPLES

The following examples are set forth below to illustrate the compounds, compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Identification of Novel Therapeutic Agents for Treating Alzheimer's Disease

Alzheimer's disease (AD) currently afflicts 5.3 million people in the United States alone. Outside of symptomatic treatment, no clear therapeutic options are available for AD patients. Conventional drug discovery paradigms are ill-equipped to combat a disease as complex as Alzheimer's disease. The systematic Alzheimer's disease drug repositioning (SMART) disclosed herein provides a systems biology paradigm to identify known drugs that could prevent or more effectively treat AD and provides a powerful, cost-effective drug discovery tool for neurodegeneration in general. By intelligently screening and matching a large number of compounds that have already been assessed toxicologically and pharmacokinetically, this systematic drug repositioning strategy significantly reduces the cost of AD drug development, enables faster-to-market clinical studies, and can identify new disease mechanisms.

In this example, initial efforts have focused on identifying existing bioactive compounds for novel uses including regulating tau phosphorylation and for use as therapeutic treatments for Alzheimer's disease. The initial compounds tested including over one thousand compounds used as drugs¹ and thousands of compounds widely used in biological research. Pharmacodynamic and pharmacokinetic properties of many of these drug compounds are well characterized. In addition, as the substrate-protein interactions of the compounds are well characterized, effective compounds can be used as probes to gain an in-depth understanding of the complete repertoire of signaling pathways underlying neuroregeneration^{2,3}.

Disclosed herein is a novel therapeutic application of a new 3D human neural culture model of AD for drug screening. While the Alzheimer's Aβ hypothesis posits that excess accumulation of Aβ is sufficient to trigger AD pathogenic cascades, current Aβ mouse models fail to fully recapitulate pathogenic hallmarks of AD, including Aβ-driven neurofibrillary tangles (NFT) and neurodegeneration. The 3D culture model of AD described herein so far is the only AD model that recapitulates both Aβ plaques and Aβ-induced tau hyperphosphorylation plus NFTs.^{4,5} Only triple transgenic mice expressing mutant forms of human amyloid-β precursor protein, presenilin, and tau develop both plaques and tangle pathology in brain tissues.⁶ However, the tau pathology in this model is mainly attributed to a tau mutation associated with familial frontotemporal lobar dementia (FTLD). The 3D AD cell culture model disclosed herein is used as a novel drug screening platform to search for AD drugs that can prevent relevant Aβ-driven pathogenic cascades, which lead to tau hyperphosphorylation, NFT, and neurodegeneration.

Finally, this example investigates how big omics databases can be repurposed for studying AD and identifying novel targets and drugs. This example takes advantage of the large, genome-wide databases recently assembled and made available through NIH-funded projects. High-throughput omics profiling has enabled the characterization of cellular response to large-scale perturbations. Libraries of biological states generated by chemical treatments have been built and continue to expand. Prominent examples are the Connectivity Map (CMAP) program^{7,8}, and its successor in the Library of Integrated Network-based Cellular Signatures (LINCS) program.^{9,10}

The SMART framework disclosed herein has many innovative aspects for Alzheimer's drug repositioning. First, this example shows the first high-throughput 3D AD-in-a-dish phenotypic screening platform by adopting a multi-well cell culture format maintained by automatic microplate washer/dispenser. The impact of candidate compounds on AD pathogenesis were directly tested by measuring pathological Aβ/p-tau aggregates and synaptic/functional deficits, which has not been feasible with other AD drug screening systems.

Next, the systematic Alzheimer's disease drug repositioning (SMART) framework integrates experimental and computational biology methods systematically with public transcriptomic profile data to enable fast-track identification and confirmation of novel drug candidates for AD therapy. Thus, several known drugs have successfully been repurposed for clinical trials in cancer¹¹⁻¹⁴, including an ongoing Phase II trial evaluating the efficacy of an old malaria drug, chloroquine, for metastatic and triple negative breast cancer.^{15,16}

The SMART framework adopts an Artificial Intelligence (AI)-based mechanism discovery scheme using deep learning to handle multi-scale big data resources covering transcriptomic profiles, phenotypic changes, and pharmacology information, uncovering novel mechanisms underlying the phenotype of interest. The drug predictions made by the combined bioinformatics and phenotypic screening approaches are tied closely to behavioral and pathological studies in animal models.

Finally, while this example focuses on identifying single known drugs targeting the Alzheimer's pathological Aβ/p-tau pathway, SMART is a generalizable drug repositioning and discovery framework that allows the neurodegenerative research community to integrate additional big data drug/compound databases, to incorporate new assays other than Tau or Aβ, e.g. mitochondria and inflammation, and to extend to other neurodegenerative diseases with different targeted assays. By providing mechanistic insight, the framework can derive synergistic drug combinations by combining drug candidates targeting different aspects of AD pathogenesis in the future.

The cell lines and methods disclosed in this example provide an effective method for the in vitro screening of compounds specifically targeting the tau pathology in AD condition. In the past, tau cell models were created by treating P301S tau overexpressing primary neurons with pre-formed tau fibrils (Guo J L and Lee V M Y, FEBS Lett. 587:717, 2013). The limitations of this previous model include (1) it is not related to the amyloid β biochemistry; (2) tau pathology does not appear naturally during cell development; (3) it requires primary cultured cells from transgenic animals so that the cell quantity is very limited, and therefore its application in high-throughput drug screening is also very limited. In addition, methods for automatically processing the tau images in such a cell model in high-throughput manner, and for analyzing the screening results and hit prioritization have not been reported.

The screening and analytic pipeline herein provides a systematic solution to overcome these limitations and address the challenges in tau compound screening. First, it uses a neural stem cell overexpressing the mutations of amyloid β genes, so that tau pathology develops gradually during cell development as a result of amyloid β biochemistry. The cells can be expanded in vitro to provide an unlimited cell source for drug screening. The tau images from the screen are automatically processed with the image processing programs and the screen hit analyzed through the bioinformatics tools. The whole pipeline provides a complete solution that has not been realized before, for the effective drug discovery on AD specific tau pathology.

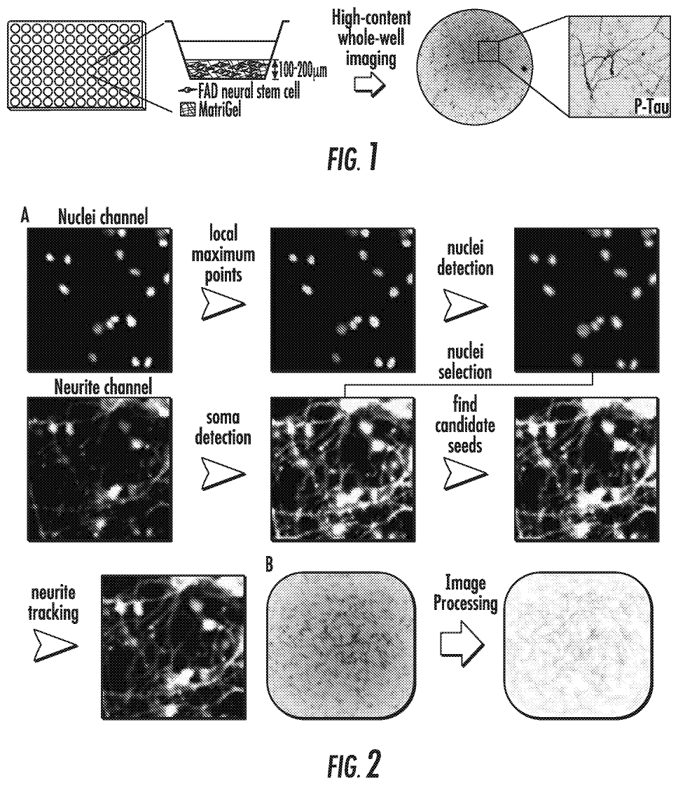

A 3D human neural culture model of AD was developed by culturing ReNCell VM cells carrying the APPSL mutation in a thin layer (50˜100 μm thick) of Matrigel. This method was then miniaturized to 96, 384, or other high content well plate formats (FIG. 1) to provide a faithful model for testing the effect of compounds on AD. However, the high-throughput screening is limited by its readout of microscopy images, which cannot be analyzed manually in large quantity.

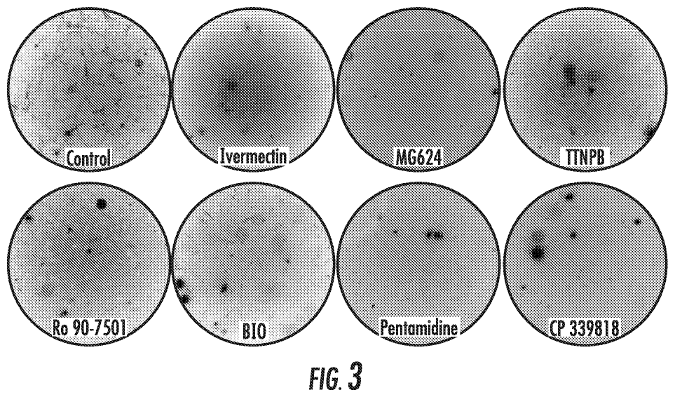

To enable high-throughput in vitro screening, an image-processing program was developed, based on NeuriteIQ software¹⁷⁻²³ (FIG. 2A), to automatically quantify tau phosphorylation from images of cultured cells. The program processes images from th e nucleus and neurite channels separately. In the nucleus channel, nuclei are detected and segmented by local maximum detection and watershed method. In the neurite channel, phospho-tau stained cells are treated as two-dimensional curvilinear structures and processed based on the local Hessian matrix^{24,25}, which allows the detection of center points and local directions of neurites in a field. The program reliably quantified the number of neurons with hyper-phosphorylated tau (FIG. 2B). Thus, a high-content screening system for the identification of compounds targeting the tau pathology in AD was established.

Known drug and bioactive compounds (2,640 compounds) were selected for the initial screen from the Sigma-Aldrich LOPAC (1280), Tocriscreen Total Library (1120), and a manual selection of 240 Kinase Inhibitors. Of these 2,640 compounds, 38 significantly reduced tau hyperphosphorylation (Table 5). Three of these 38 hits, ivermectin, MG624, and pentamidine, almost completely inhibited pTau, with no visible fibrous structure left in the well (FIG. 3). A few of the 38 compounds have been previously reported to inhibit tau phosphorylation in AD animal models (e.g., baicalein²⁶ and tretinoin²⁷), further validating the credibility of the screen. Most, however, are novel AD candidates.

TABLE 5
Compounds identified in the preliminary screen and previously known functions

| Name | Previously known function |
|---|---|
| Ivermectin | Positive allosteric modulator of a7 nChR |
| MG 624 | α7 nChR antagonist |
| Ro 90-7501 | Inhibitor of Aβ42 fibril formation |
| BIO | Potent, selective GSK-3 inhibitor |
| CD 1530 | Potent and selective RARγ agonist |
| Retinoic acid | Endogenous retinoic acid receptor agonist. |
| TTNPB | Retinoic acid analog; RAR agonist |
| Loperamide hydrochloride | Peripherally acting μ agonist. Also Ca2+ channel blocker |
| Mibefradil dihydrochloride | Ca2+ channel blocker (T-type) |
| Nifedipine | Ca2+ channel blocker (L-type) |
| NNC 55-0396 dihydrochloride | Highly selective Ca2+ channel blocker (T-type) |
| RS 67333 hydrochloride | 5-HT4 partial agonist |
| SB 206553 hydrochloride | Potent, selective 5-HT2C/5-HT2B antagonist. Orally active |
| Y 29794 oxalate | Prolyl endopeptidase inhibitor |
| WIN 64338 hydrochloride | Bradykinin B2 antagonist |
| U-75302 | BLT1 leukotriene receptor agonist |
| SB 408124 | Selective non-peptide OX1 antagonist |
| RS 17053 hydrochloride | α1 A antagonist |
| Rottlerin | Inhibit PRAK and MAPKAP-K2 |
| Rapamycin | Specific inhibitor of mTOR |
| PP1 | Potent inhibitor of Src-family tyrosine kinases |
| PHA 665752 | Potent and selective MET inhibitor |
| Pentamidine isethionate | NMDA glutamate receptor antagonist |
| LFM-A13 | Potent, selective BTK inhibitor |
| Leflunomide | Dihydroorotate dehydrogenase inhibitor |
| JK 184 | Hh signaling inhibitor; alcohol dehydrogenase 7 inhibitor |
| Fluticasone propionate | Selective high affinity glucocorticoid agonist |
| Ebselen | Mammalian lipoxygenases and GST inhibitor |
| Cyclosporin A | Calcineurin inhibitor |
| CP 339818 hydrochloride | Non-peptide, potent KV1.3 channel blocker |
| CHR 2797 | Aminopeptidase inhibitor |
| CH 223191 | Potent aryl hydrocarbon receptor (AhR) antagonist |
| CGP-74514A hydrochloride | Cdk1 inhibitor |
| Bromoacetyl alprenolol menthane | Alkylating beta adrenoceptor antagonist |
| Baicalein | 5- and 12-Lipoxygenase inhibitor |
| Arcyriaflavin A | Potent cdk4/cyclin D1 and CaM Kinase II inhibitor |
| Actinonin | Leucine aminopeptidase inhibitor |
| 1,4-PBIT dihydrobromide | iNOS inhibitor; eNOS inhibitor |

### Example 2. Workflow for the Systematic Alzheimer's Disease Drug Repositioning (SMART) Framework

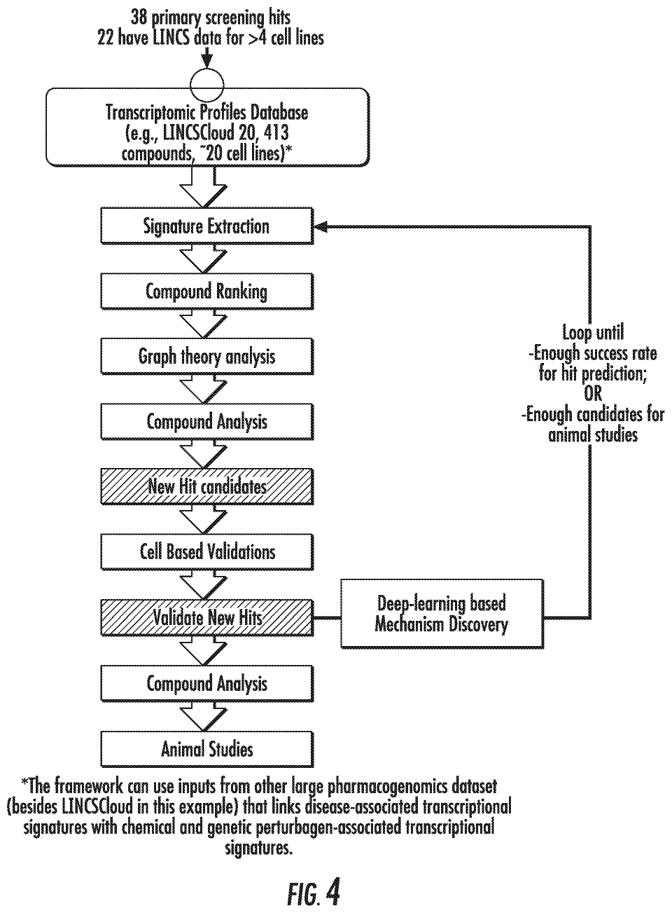

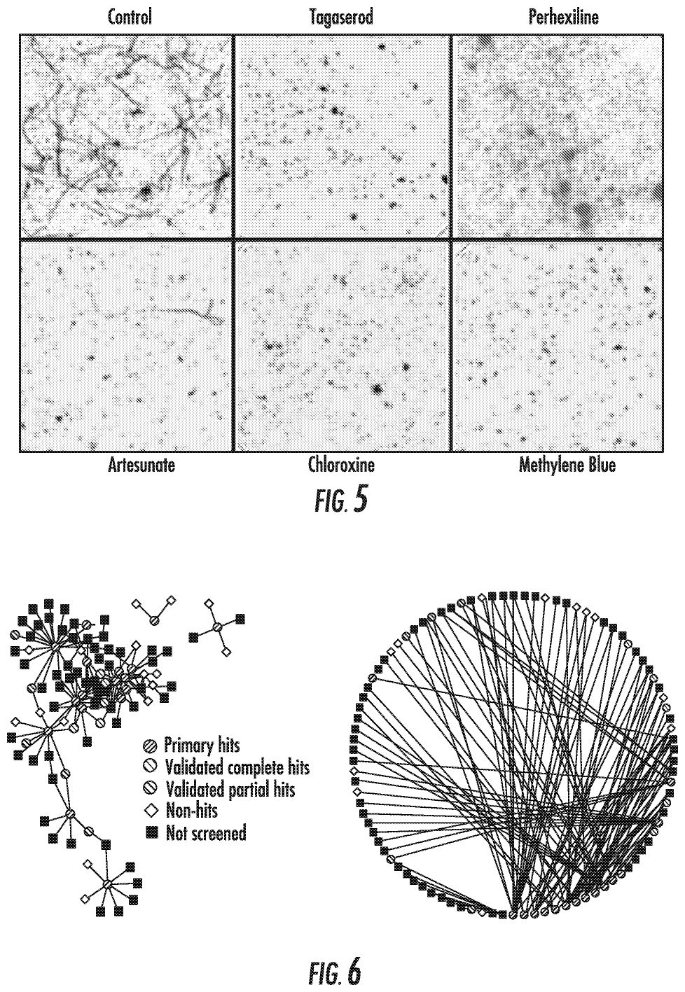

In this example, an iterative and integrative screening workflow in the systematic Alzheimer's disease drug repositioning (SMART) framework for drug repositioning was developed (FIG. 4). This bioinformatics-driven workflow leverages publicly available large transcriptomic profiles of cellular responses to various perturbations, especially small molecular compound treatments. These I/O and analytic strategies ensure that public or in-house transcriptomic profiles generated using different technologies and platforms, e.g., RNAseq and microarray, are seamlessly incorporated. The signature extraction step serves as the interface for accepting feedback information flow and initiating new loops. The first iteration starts with signatures covering the whole genome, and the results undergo cell assay validations and expand the training sets of hits vs. non-hits for deep learning based mechanism discovery, ultimately leading to a refined signature consisting of phenotype-related pathways.

Subsequent iterations start with a signature focused on pathway changes correlated to phenotype changes of interest, improving predictions of candidates for new hits.

As a proof of concept, the transcriptomic profiles hosted by the Broad Institute's LINCSCloud data warehouse²⁸⁻³⁰ through the NIH LINCS program were used in the initial study. The LINCSCloud dataset covers ˜20 cell lines' response profile to 20,413 small molecule compounds, including ˜1,300 FDA approved drugs and more than 5,000 bioactive compounds and experimental and shelved drugs.

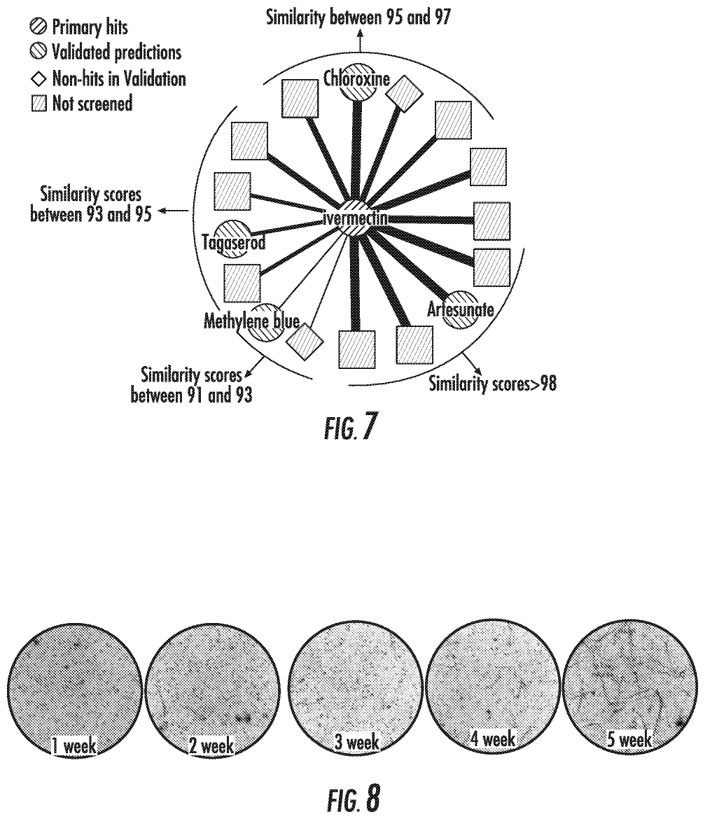

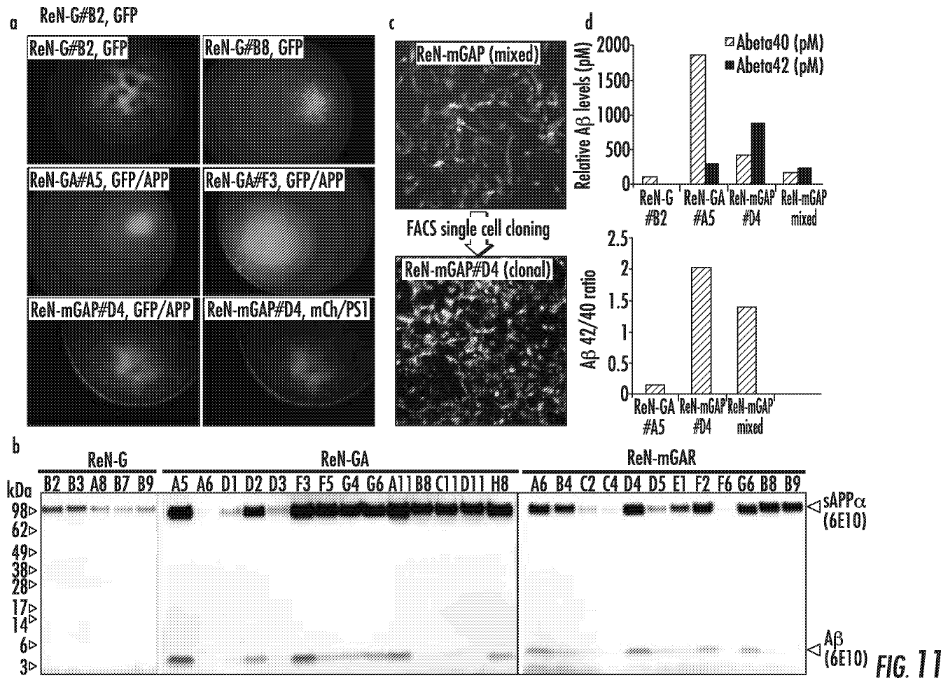

Twenty-two of the 38 aforementioned screening hits had LINCS data covering the perturbation profiles for at least 4 cell lines. From these 22 hits, 2 were eliminated because no known drug candidates ranked high enough based on transcriptomic similarities to these two primary hits; and 3 others were removed upon inspection of the compound properties of the predictions they made, i.e., the predicted drugs may be toxic or unfit for systematic use. Thus, the 17 primary hits shown in Table 1 were used to initiate a pilot run using the SMART framework. The cMAP algorithm⁷ was used to rank all compounds in the LINCSCloud, based on the similarity of transcriptomic profiles to each of the 17 primary hits. If any compound was determined by cMAP algorithm to have a similarity score larger than 90 to at least one of the primary hits, it was identified as a hit candidate. After filtering based on pharmacology features, 85 candidates predicted by 17 primary hits (Table 1) remained; 26 of these 85 compounds were purchased for validation after analysis for pharmacology and medical practice features. According to the validation results, 10 of these predictions significantly inhibited pTau (See Table 2, Table 6). Five compounds almost completely inhibited pTau in the reformatted high content version of AD-in-a-dish model (with compound names listed in FIGS. 5 and 7), achieving phenotypes comparable to those from the top-3 hits (ivermectin, mg624, and pentamidine) in the primary screen.

TABLE 6
Compounds identified in the SMART screen and previously
known functions

| Name | Previously known function |
|---|---|
| tegaserod | to treat irritable bowel syndrome and constipation |
| maleate | |
| perhexiline | approved in Australia and New Zealand as a prophylactic |
| maleate | antianginal agent |
| liothyronine | to treat hypothyroidism and myxedema coma, also used |
| sodium | as augmentation agent to treat major depressive disorder |
| dasatinib | a cancer drug to treat chronic myelogenous leukemia and |
| monohydrate | Philadelphia chromosome-positive acute lymphoblastic |
| | leukemia |
| pazopanib | a cancer drug to treat renal cell carcinoma and soft tissue |
| hydrochloride | sarcoma |
| vemurafenib | to treat BRAF V600E mutation positive unresectable or |
| | metastastic melanoma |
| olaparib | a cancer drug to treat ovarian, breast, and prostate |
| | cancers with hereditary BRCA1 and BRCA2 mutations |
| artesunate | an antimalarial drug |
| methylene blue | mainly used to treat methemoglobinemia, also used |
| | as a dye |
| chloroxine | an antibacterial drug to treat infectious diarrhea, intestinal |
| | microflora disorders, giardiasis, and inflammatory bowel |
| | disease |

Even without further iterations, this smart drug screening workflow achieved a 5.88% ( 5/85) success rate in predicting hits, more than a 51-fold improvement over the 0.114% ( 3/2640) hit identification rate of the primary screening.

Novel computational algorithms are developed for the key steps of signature extraction, compound ranking, and graph-theoretical analysis (dotted-line box of FIG. 4). The results from cell-based validation and mechanism discovery are fed back to modify the signature extraction step, with the goal of providing more accurate target signatures for compound ranking in a new iteration, initiating an iterative workflow to improve the success rate for hit prediction and expand the group of repurposed drug candidates for AD that are validated by animal studies.

Signature Extraction

The pilot run used the cMAP algorithm for compound ranking, which summarizes the expression signature for each compound treatment using genes with the top 100 and bottom 100-fold expression changes under control conditions. This scheme may be over-simplified in that it is vulnerable to expression profile outliers while the fixed cut-off number for significant genes may lead to ignorance on certain key expression changes and thus underestimation of the global picture of pathway activities.

For more robust signature extraction in the SMART framework, Gene Set Enrichment Analysis (GSEA)^{28,31} is used to transform the transcriptomic data into a series of enrichment scores for functionally related gene sets. For the expression profile of each compound, GSEA provides enrichment scores for up to 13,000 gene sets defined in the MSigDB database²⁸. The scores from categories C2.CP (1,330 canonical pathways covering databases including KEGG^{32,33} BIOCARTA^{34,35} and REACTOME^{36,37}), C3 (836 motif gene sets³⁸ covering targets of miRNA and transcription factors³⁹), C5 (1454 Gene Ontology^{40,41} terms covering biological process, molecular function, and cellular compartment), and H (50 hallmark gene sets defined by the MSigDB database⁴²) are used. The compound perturbation omics signature is compressed into ˜3,620 enrichment scores. This new signature extraction scheme facilitates inclusion of transcriptomic profiles generated by other technology and platforms, as GSEA generates signatures of equal size after platform-specific processing within each dataset.

Compound Ranking

To measure the similarity between target signatures from compounds i and j from LINCSCloud, we will generate a combined score incorporating the similarities between their perturbation profiles and chemical properties. The similarity metric proposed in⁴³ will be combined with the metrics in the STITCH database⁴⁴ to quantify the similarity between two compounds i and j. After GSEA analysis, the similarity metric S_{G} (i, will be defined as the Pearson Correlation Coefficients between the two vectors. An additional similarity metric, Sₛ(i,j) will be defined based on the STITCH database⁴⁴ by integrating a combined score of the structure similarity and text-mining similarity score. The structure similarity is defined by the Tanimoto 2D chemical similarity scores⁴⁵ while the text mining similarity is computed by mining a curated database, such as OMIM⁴⁶ and MEDLINE, using a co-occurrence scheme and a natural language processing approach^{47,48}. The two similarity metrics combined as: S(i,j)=αSₛ(i,j)+S_{G} (i,j), j=1, 2 . . . 20,413, where α is the parameter controlling the level of emphasis for structure information. Here, each target compound i corresponds to one of 17 primary hits in our pilot run, and for each i, there are 20,413 similarity scores that can be normalized into Z-scores. Top-ranked compounds with p-value <0.05 are selected as candidate hits.

Graph-Theoretical Analysis:

In each iteration of the SMART screening workflow, the relationships among target compounds, predicted hit candidates, and validated hits will be modeled using a directed graph (DG) model⁴⁹. After compound ranking, each target compound i is associated with a group of predicted compounds Pᵢ={pᵢ^{(x)}}, x=1, 2 . . . m, which are selected based on the cut-off on compound similarities. A directed graph G=(V,E) can then be defined, with the set of vertices V=I∪P, where I={1, 2 . . . n} is the set of target compounds and P={P₁, P₂ . . . Pₙ} is the set of predicted compounds. In our pilot run, the set of target compounds is the group of primary hits with LINCS data; thus n=17 and the size of P is 85. Meanwhile, the set of edges, E only includes directed edges in the form of e={i, pᵢ^{(x)}}, with weight on the edge wₑ=S(i,pᵢ^{(x)}), i.e., each edge will always be from one target compound to one of its predicted compounds, with the similarity between two connected compounds serving as the edge weight.

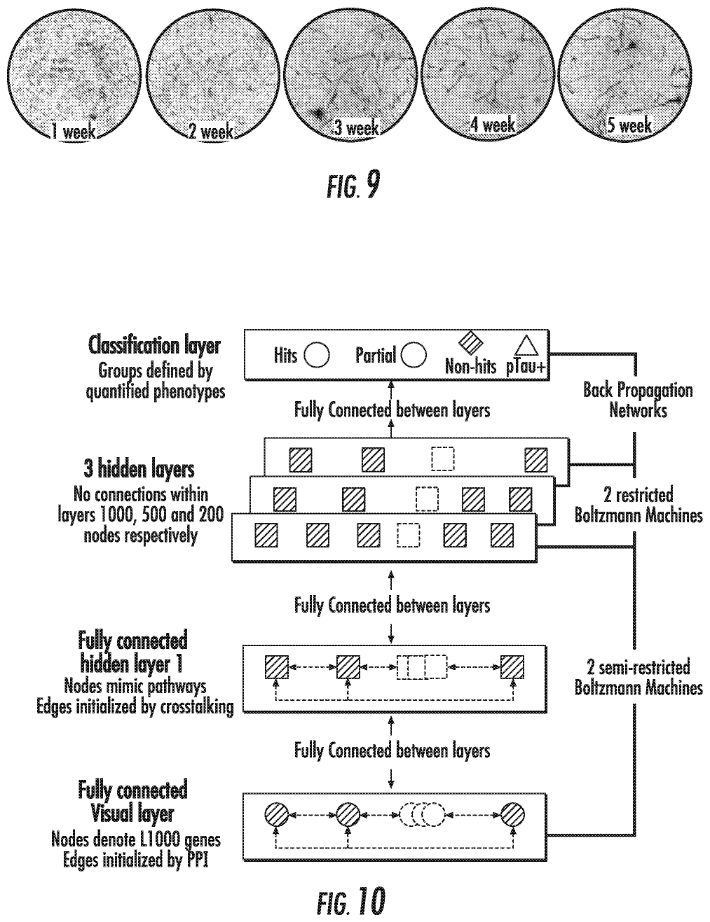

FIG. 6 summarizes the results for the pilot run: 17 primary hits (blue nodes) connected to 85 predicted compounds (yellow, green and gray) through a total of 215 edges, the thickness of the edge is proportional to the edge weight. Three isolated communities exist in the graph: one of the primary hits, Ro90-7501, forms one isolated community with its four predictions; another primary hit, TTNPB, forms another community with its two predictions. The remaining nodes form the largest connected community. FIG. 6 also shows that connected community in a degree-sorted circular view: a total of 94 connected nodes (15 primary hits and 79 predictions) are positioned in a circle, with the compound having the most neighbors located in the six o'clock position and all other nodes located in counter-clockwise order with descending degrees. This view reveals that 14 out of 17 primary hits have a degree larger than 7; also, 4 of 5 (yellow) validated hits have a degree larger than 4, ranking them among top 18 out of all 85 predicted compounds (chloroxine in FIG. 5 has a degree of 3 and ranked 22nd); meanwhile, all 5 (green) partial hits have a degree no more than 2.

In addition to the above “big picture” analysis of the overlap between predictions made by multiple target compounds, DG is also used to assess the relationships between individual target compounds and its predictions. Ivermectin has the most significant phenotype of the 38 primary hits (FIG. 7), and 4 of 5 successful predictions (except for Perhexiline in FIG. 5) in the pilot run have similarity scores larger than 90 with ivermectin. Of the 16 compounds predicted by ivermectin, 10 (gray squares) were not purchased after analyzing their previous medical usages. Thus 4 out of 6 (66.7%) ivermectin predictions tested were validated, much higher than 5.88% for the pilot run overall. By comparing with FIG. 6, Artesunate and Chloroxine have similarity scores larger than 95 in FIG. 7, yet their overall degrees are smaller than those of compounds Tegaserod and Methylene Blue.

This study revealed specific graph-theoretic characteristics for the validated hits from the pilot run. Thus, more validated hits can be revealed with more iterations of the workflow, these validated hits serve as cluster centers and divide the whole space of 20,413 compounds into highly connected clusters, and the validated hits are enriched in these compound clusters such that it is possible to predict hit compounds within certain clusters based on the graph-theoretic features, e.g. yellow nodes among the largest community in FIG. 6 mostly have larger degrees.

The graph in FIG. 6 is expanded using the nodes brought in by future iterations of the workflow. A series of graph-theoretical features, e.g., the panel of eighteen features⁵⁰, are calculated for each node. These features represent different aspects of graph-theoretical properties. Features like clustering coefficient⁵¹ and information centrality^{52,53} for each validated hit are incorporated with hierarchical clustering methods to divide the connected part of the graph into highly connected or highly centralized sub-graphs. Within each sub-graph, SVM classifiers⁵⁴⁻⁵⁶ are trained to differentiate validated hits vs. non-hit compounds based on their graph theory properties. When a new compound is introduced to the graph, it is assigned to one of the pre-defined sub-graphs based on its similarity with known hits, and its graph theory features is fed into the specific classifier for this sub-graph to generate a confidence score as to whether this compound tends to have similar graph features as those known validated hits in the same sub-graph.

Compound Feature Analysis:

After unbiased ranking of all 20,413 compounds by their transcriptomic similarity to each target compound, a series of filtering procedures are applied based on the features of top-ranked compounds. First, confirmed non-hits, i.e. compounds that failed to show significant phenotypes in previous screening or validations, are eliminated. Remaining compounds are assigned into four categories: approved drugs, clinical trial drugs, investigational compounds, and compounds with limited information.

In some examples, the focus is on finding novel AD therapies, and only approved drugs (currently approved by FDA, discontinued, or internationally approved) or clinical trial drugs are kept as candidates for repurposing.

These candidates are filtered by pharmacological features and other practical considerations including toxicity (drugs requiring Health Safety Committee (HSC) review based on GHS Cat.1⁵⁷ are eliminated), systemic usage (drugs not approved for systemic usage are eliminated), and commercial availability.

Iterative Running of Functions Using Feedback Information Flow:

As shown in FIG. 4, all functional modules defined above run iteratively to effectively search the space of all available compounds, find new screening hits, and ultimately provide candidates for novel AD therapy. Feedback information flow is used to control both the width and depth of the search scheme. Refining the number of bait compounds and modulating signature content and help control the search width. Specifically, given the panel of predicted compounds from any iteration, 3D-cell based validation assays assure that only true hits corresponding to significant phenotype changes serve as the “baits” for the next iteration.

Meanwhile, based on the validation results, all predicted compounds are added to the training sets of hits vs. non-hits, allowing the deep-learning workflow to gain a better understanding of transcriptomic features underlying phenotype changes of interest. The output of the deep-learning analytics in the SMART framework consist of a series of key pathway changes, which can then help refine the content of transcriptomic signatures used in the next iteration, allowing the search scheme to focus on key pathways that continuously generate validated predictions. The depth of this workflow is correlated to its efficacy; specifically the success rate of hit prediction overall and within each iteration. The iterative workflow can be terminated when enough (5-10) novel drug candidates are collected for animal studies or when the updated mechanism information brings the success rate of hit prediction to a desirable level (for example, over 75%).

Constructing a Deep Learning Workflow to Uncover the Molecular Mechanisms Underlying Compounds that Block AD Pathogenic Events.

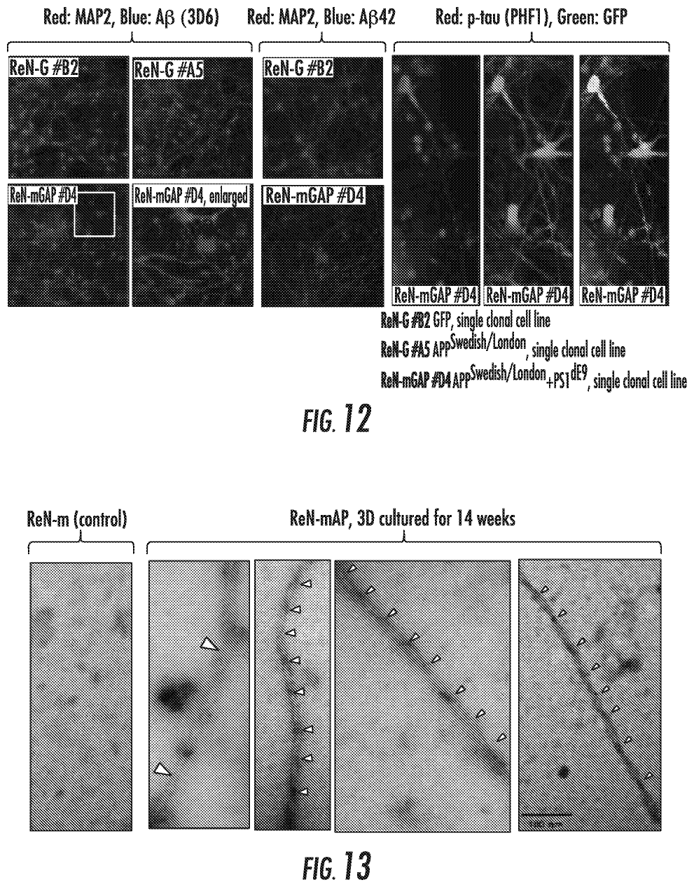

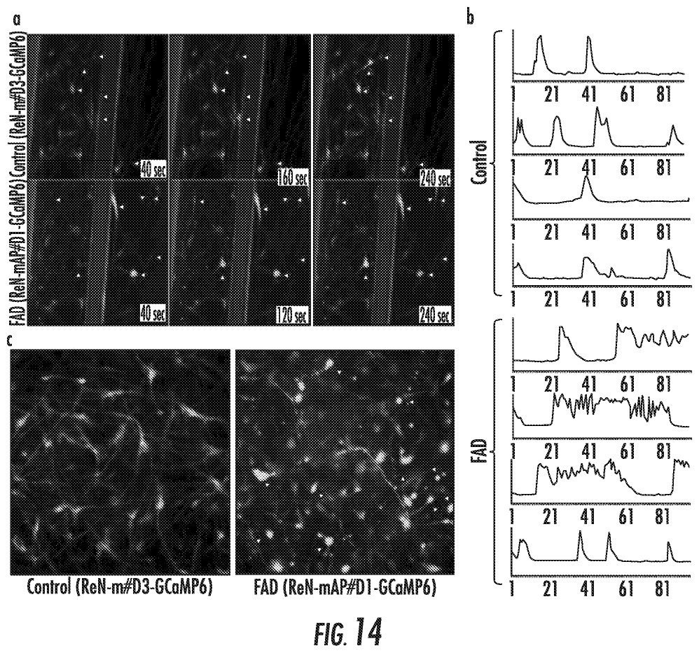

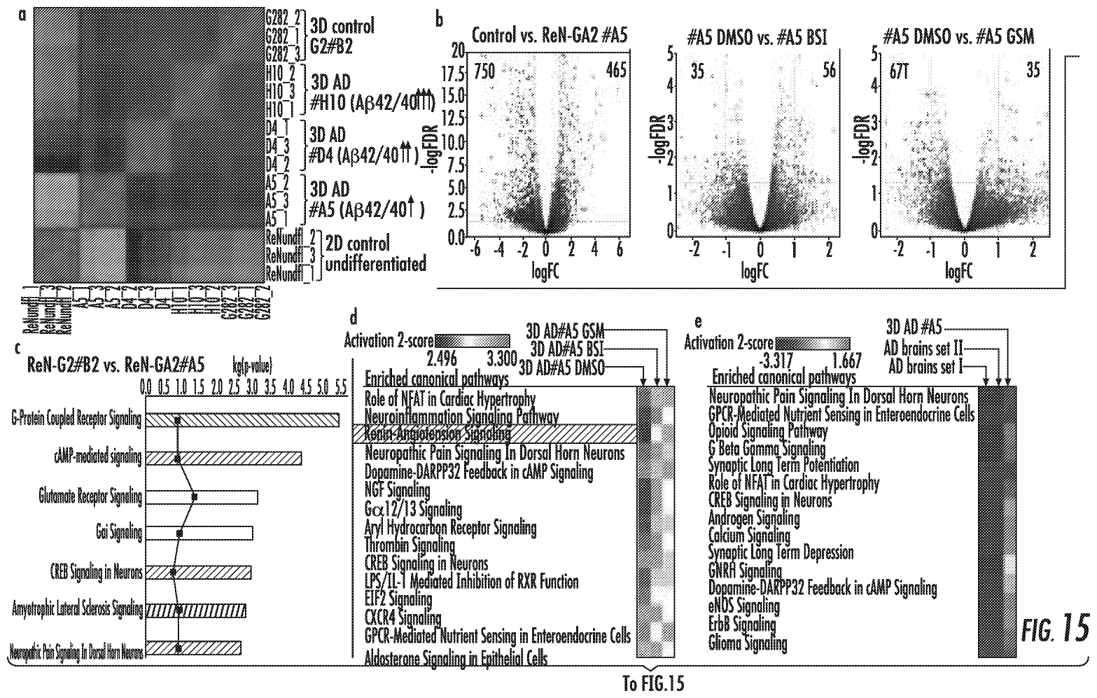

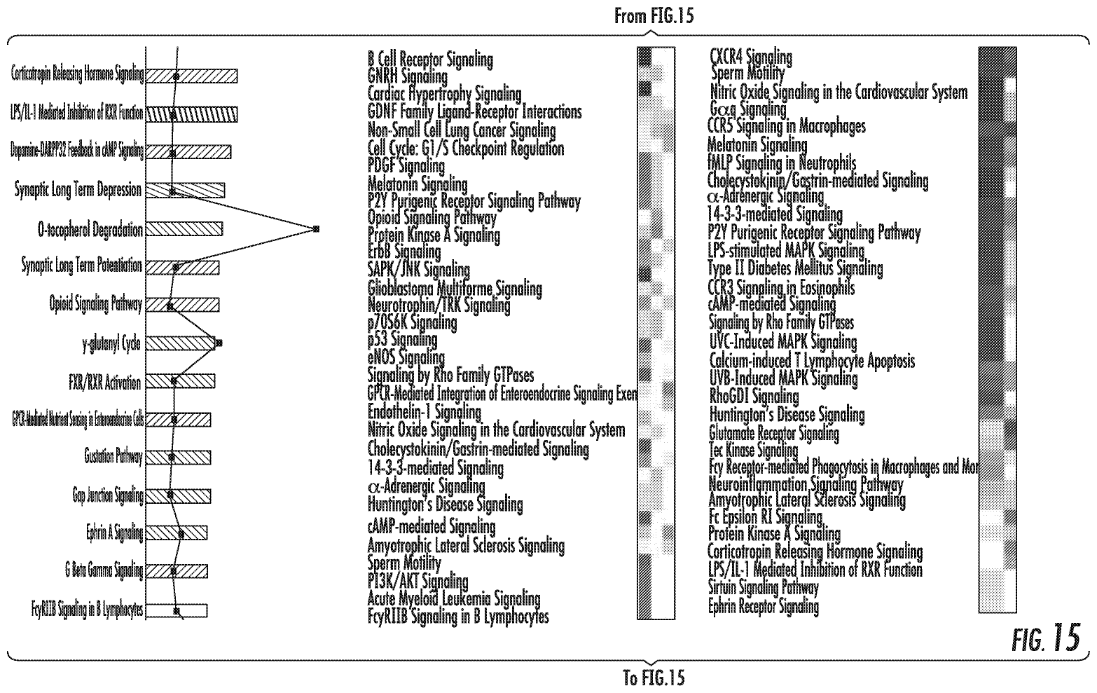

A number of bioactive compounds that were identified in the 3D phenotype screen exhibited highly interesting properties and can be used for studying disease mechanism and identifying therapeutic drugs. The primary screening hit compounds reduced tau phosphorylation when added to cells from the beginning of culture. Notably, tau phosphorylation in the neurites developed gradually during stem cell neuronal differentiation (FIG. 8), appearing after two weeks of culture and gradually increasing until week 4.

After that, high levels of tau phosphorylation were maintained in the neurites. Several compounds, e.g., MG624, significantly reduced tau phosphorylation when added after week 2, when tau phosphorylation should have already developed. In fact, when the compounds were added after four weeks when tau phosphorylation was already maximized in neurites, the compound still reduced p-tau after two weeks of treatment (FIG. 9). This shows that these compounds either reverse tau phosphorylation or selectively eliminate cells with tau hyperphosphorylation, highlighting the importance of further mechanism study of the compounds.

Clustering Analysis Reveals Shared Mechanisms Among Confirmed Hits:

The SMART screening framework incorporates publicly available transcriptomic profiles with the 3D AD-in-a-dish model. As more predicted hits are confirmed through the 3D cell assay, more light is shed on novel pathways and mechanisms possibly underlying the phenotype of interest, i.e., inhibition of pTau. Nineteen transcriptomic profiles were obtained from LINCSCloud where confirmed hits from this assay (including part of 17 primary hits and members of 5 validated hits from the pilot run) were applied to the NEU adult neuron cell line. Gene Set Enrichment Analysis was applied to each profile to generate enrichment scores for 186 canonical pathways defined in the KEGG database. Two-way hierarchical clustering⁶¹⁻⁶³ using centroid linkage with Pearson correlation coefficients (PCC) as the similarity metric was applied to the panels of enrichment scores for all 19 compound treatments. Under the cutoff of PCC>0.80, the 19 compound treatments can be divided into two clusters under proper cutoff. Consistent with the graphs in FIG. 6, the center of a disconnected community, TTNPB, and the center of an isolated sub-graph, PP1, form a smaller cluster; while the larger cluster features highly connected nodes such as rottlerin and loperamide, as well as hits such as chloroxine in FIG. 5, which was discovered by the SMART framework. Several sub-groups among the 186 KEGG pathways show significant changes corresponding to the compound treatments covered in this clustering analysis; a few show opposite trends between two sub-groups of compounds defined above.

Specifically, KEGG pathways related to Alzheimer's, Parkinson's, and Huntington's disease, which are enriched with mitochondria-related genes, largely went down with the cluster of rottlerin and chloroxine, etc, and went up with the cluster featuring TTNPB and PP1. Meanwhile, pathways related to long-term depression, focal adhesion, and MAPK signaling, among others, show an opposite trend and went up with the rottlerin-chloroxine cluster.

Deep Belief Networks (DBN) for Identifying Mechanisms Underlying pTau Regulation:

As the iterative workflow proceeds, more compounds have matched transcriptomic and phenotypic profiles to show whether they effectively regulate pTau. A deep learning based AI model using DBN is developed to: 1) use unsupervised deep learning to understand the regulatory structure of transcriptome data, and 2) incorporate class labels defined from quantified pTau phenotypic profiles to identify gene modules underlying pTau regulation. Level-4 differential expression profiles from LINCSCloud is also used.

The planned DBN is a stacked neural network with six layers (FIG. 10). The bottom five layers (named overall-visible layer and hidden layers 1-4, respectively, from bottom up) accomplish the unsupervised deep learning by forming four restricted Boltzmann machines (RBM). The top layer includes group labels defined by cell-based validations, e.g. confirmed hits, partial hits, non-hits, and even increased pTau. It is used to adjust parameters in the lower levels in back propagation (top-down) style. Each node from the lowest layer corresponds to individual gene expression levels measured for each L1000 landmark gene; the nodes learned from hidden layer 1, whose values are determined jointly by nodes in the visual layer, can be interpreted as gene modules. The values of nodes in hidden layers 2-4 are determined jointly by the nodes in the immediate lower layer, and thus potentially reveal higher order regulatory and crosstalk mechanisms among gene modules.

An RBM consists of a layer of visible variables vᵢ, i=1, . . . , m, and a layer of hidden variables hᵢ, j=1, . . . , g. The nodes are fully connected across two layers, with no connection allowed within the same layer. Let symmetric matric W=(w_{i,j})_{m×g} represent weights between two layers of variables, while a=(a₁, . . . , aₘ) and b=(b₁, . . . , b_{g}) represent bias vectors corresponding to each variable in visible and hidden layers, respectively. Given a joint configuration (v, h) for the RBM, an energy function of an RBM model can be defined for binary visible and hidden unit as E(v, h; θ)=a^{T}v+b^{T}h+v^{T}Wh, with θ=(a, b, W). In our case, hidden layers 2-4 are composed of binary units while the overall visible layer consists of random variables following Gaussian distributions (because level-4 data are Z-scores), which corresponds to the expression profile of m=978 landmark genes measured in the Broad Institute L1000 protocol. For the RBM involving overall visible layer and hidden layer 1, the energy function is rewritten as:

$$E ( v , h ; θ ) = {\sum }_{i = 1}^{m} \frac{{( {v}_{i} - {a}_{i} )}^{2}}{2 {σ}_{i}^{2}} + {\sum }_{i = 1}^{m} {\sum }_{j = 1}^{g} \frac{{v}_{i}}{{σ}_{i}} {w}_{ij} {h}_{j} + {\sum }_{j = 1}^{g} \frac{{v}_{i}}{{σ}_{i}} {b}_{j} {h}_{j} .$$

Either way, the probability density function of a joint configuration (v, h) can be defined as

$$f ( v , h ; θ ) = \frac{1}{Z ( θ )} exp ( - E ( v , h ; θ ) ) ,$$

with conditional density distribution defined accordingly. Correlations among input variables are allowed as the learning procedures canceling the correlations out.⁶⁴

In our case, the overall visible layer has m=978 while hidden layer 1 is allocated 3,000 nodes, comparable to the combined number of canonical pathways (1330) and GO terms (1454) in the MSigDB database⁶⁵. W=(w_{i,j})_{m×g} between these two layers is initialized to reflect the gene set membership, i.e., w_{i,j}=1 if gene i belongs to gene set (pathway or GO term) j according to the MSigDB. This weight is bound to change according to the data structure during the learning steps, reflecting the pathway rewiring effects of gene mutations in cancer cell lines. Hidden layers 2-4 are planned to have 1,000, 500, and 200 nodes, respectively, to uncover the hierarchical structure and crosstalk among gene modules.

Currently, we have more than 1,600 compounds with matched transcriptomic profiles and phenotype labels (>50% of the 2,640 compounds in primary screening have transcriptomic profiles in LINCSCloud, and the pilot run gave phenotypic labels to 26 predicted compounds, confirming 5 as hits) that will be used to learn the DBN parameters using contrastive divergence −k (CD-k) algorithms⁶⁴. Each RBN is trained greedily with the change of weight given by: Δwᵢⱼ=δ( [symbol] vᵢhᵢ [symbol] _{data}− [symbol] vᵢhᵢ [symbol] _{reconstruction}), with δ the learning rate and [symbol] vᵢhᵢ [symbol] _{data} the fraction of time the i-th visible unit and hidden unit are simultaneously on when the hidden units are driven by training data. [symbol] vᵢhᵢ [symbol] _{reconstruction} is the corresponding fraction when the hidden layers are reconstructed after k rounds of Gibbs sampling^{66,67}.

The CD-k algorithm approximates the result of maximizing the log likelihood function of the data by minimizing the Kullback-Leibler divergence and has been proven useful in many cases, even with k=1. The learning of our DBNs will be carried out on the computer cluster in the Houston Methodist Hospital Data Center. We will compare the results for k=1-5 for their performance of differentiating different phenotype groups.

### In Vitro and In Vivo Validation.

Selected compound hits are then tested in cell and animal models, and validation results provide iterative feedback to improve drug repositioning and mechanism discovery. The impact of candidate compounds on AD pathogenic cascades of (i.e. p-tau accumulations, synaptic/functional deficits, and neuronal death) is evaluated in the 3D human neural cell culture model of AD and mouse tauopathy models. The repositioned highly potent known drugs or bioactive compound candidates are then used for clinical studies.

The 3D human neural culture model of AD disclosed herein is the first to recapitulate Aβ plaque-like aggregates and robust Aβ-driven tauopathy⁴⁻⁵. The 3D models are used to fit high-throughput testing and mechanistic studies (single-clonal AD lines). In addition to assessing Aβ and tau pathology, these improved 3D culture models can be used to assess functional deficits (GCaMP6 lines) and neuronal death (data not shown). These newly improved 3D cellular AD models are used to determine if a selected compound hit can rescue functional deficits in AD 3D culture models and Aβ/tau pathology.

The Tg mouse strain bearing APP/PSEN1 and human Tau mutants (3×Tg) is the best currently available animal model that mimics tauopathy under AD-like conditions. A majority of AD neuropathological characteristics of have been documented in this strain, including aberrant APP metabolism, tauopathy, synapse damage, and cognitive impairment^{6,68}. This AD mouse strain is used to test the therapeutic effects of these drug candidates on cognitive deficits and neuropathology, including tauopathy and synapse damage.

### Example 3. Effects of AD Drug Candidates on 3D Human Neural Cell Culture Model of AD

Cell Lines:

The impact of candidate compounds on AD pathology and functional deficits are assessed in three different AD ReN cell lines with different Aβ42/40 expressions (ReN-mAP # E6F4, HReN-mGAP30, and ReN-mAPGCaMP6# D1). Control human neural stem cell lines, ReN cells expressing eGFP/mCherry, and human induced pluripotent stem cell (hiPSC) -derived neural stem cells (from ScienCell Research Laboratories) are used to test for potential toxicity under physiological conditions. These cells all exhibit robust Aβ accumulation and tau pathology.

3D Cell Culture and Drug Treatments:

Thin and thick-layer 3D cultures are generated as previously described with slight modifications^{4,5}. Thin-layer 3D cultures are plated using BioTek liquid handling systems (MultiFlo™ FX) and the culture media is changed every three days. Cultures are differentiated for three weeks and then candidate compound hits are applied for 3 additional weeks. Five different doses with 4 to 5 wells for each condition are used to validate the impact of candidate compounds. For thick layer culture, 24-well transwell inserts are used as previously described^{4,5} and treated with single or multiple doses. The toxicity of the candidate compound is consistently monitored by fluorescence microscopy and LDH release assay.

Analysis of Aβ and p-Tau Pathology:

Soluble and insoluble Aβ40/42/38, total tau, and p-tau (pSer181) levels are measured by electrochemiluminescence/multi-array technology (MSD). Immunofluorescence staining is also used to assess abnormal p-tau accumulation and mislocalization. Biochemical analyses is performed for the thick layer culture with or without drug treatments. If needed, EM imaging is performed to directly visualize Aβ and tau fibril structures before and after drug treatments.

Analysis of Functional Deficits and Cell Death:

To measure the impact of candidate drugs on functional deficits, abnormal Ca2+ influx, and hyperactivity, control and AD cell lines are used stably expressing GCaMP6 Ca2+ reporter protein (ReN-mGCaMP # D3 and ReN-mAPGCaMP # D1). Unbiased semiautomatic imaging and time-lapse imaging are performed in vivo using a Nikon Al laser confocal system. VGluT1/Synapsin 1-positive synapse-like puncta in AD cells is measured with or without candidate compound treatments.⁵ To test if the candidate drugs can selectively reduce neuronal death in late AD cultures (>9 weeks), cell survival rates are measured by using 1) LDH release assay, 2) 3D-compatible RealTime-Glo MT cell assay kit (Promega), 3) active caspase 3 staining, and 4) unbiased DAPI nuclear staining. In the preliminary studies, significant increases in neuronal death were observed in 3D-differentiated AD cells as compared to the controls (data not shown).

Some of the candidate compounds target upstream of Aβ accumulation while others block downstream of Aβ accumulation, both of which can decrease p-tau accumulation. Some of the compounds can block both Aβ and p-tau accumulation by multiple mechanisms. Depending on the mechanisms of action, these drugs can have differential effects on functional deficits and cell death. Candidate drugs may decrease both p-tau and functional deficits. Candidate compounds may decrease both Aβ and p-tau accumulations.

### Example 4. Effects of AD Drug Candidates on Neuropathology in 3×Tg Mice

Tg Mice Maintenance and Group Setting:

For testing each drug, 3×Tg homozygous (Tg 1-3) and WT control mice in 3 groups (Wt 4-6) are used (n=8/group). Mice are treated with a drug or vehicle at two time points (6 and 10 months of age) to study the dynamic change of abeta/tauopathy pathological cascades.

Drug Administration:

Drug candidates are dissolved in 0.9% NaCl. Oral gavage ingestion is used to deliver drugs daily for five weeks before the initiation of the behavioral tests and throughout the study.

The same volume of vehicle is applied to the control mice in group 1 (Tg-1) and group 4 (Wt-4). A low dosage of drug candidate is delivered to mice in group 2 (Tg-2) and group 5 (Wt-5) while a high dosage of the drug is administered to mice in group Tg-3 and Wt-6. Body weights of mice are monitored once a week.

Tissue Collection:

Tg mice raised at MGH are deeply anesthetized and perfused transcardially with ice cold PBS after experimental endpoints. Mouse brains are immediately removed and cut sagittally. For western blot, the desired brain tissues are dissected from the left brain hemisphere from five mice while the right hemisphere is fixed with ice cold 4% PFA for morphological analysis following previous methods⁶⁹. Partial cerebral cortex is freshly dissected for isolation of synaptosome following published protocol⁷⁰.

Detection of Tau Aggregates:

Tau aggregates are examined morphologically via immunostaining on brain sections, or biochemically on brain homogenates. For immunohistochemical staining, floating sections are permeabilized and incubated in blocking solution, followed with anti-tau-p (AT8, MC-1, PHF-1) or anti-total tau (Tau-5). HRP-labeled DAB-based ABC immunohistochemistry⁶⁹ are used to visualize tau aggregation in brain section. For immunofluorescence staining, AT8, MC-1, PHF-1 are used with Tau-5 to visualize tau tangles by dual labeling with Alexa Fluor 488- and Alexa Fluor 555. Gallyas silver staining is used to visualize tau tangle-like structures in brain. Three sections (4 fields each) are examined by microscopy at 400× magnification. Silver-(+) neuronal cell bodies and neurites are recorded per 0.1 mm². For western blot assays to detect Tau aggregates, AT8, MC-1, PHF-1 are used to examine p-Tau level while Tau-5 and anti-GAPDH are used for detection of total tau and internal standard.

Neurodegeneration Examination:

Synapse damage is examined by immunofluorescence staining of presynaptic (synapsin I) and postsynaptic (PSD95) proteins on brain sections as described⁷¹. Western blot is used to examine the levels of these proteins in synaptosomes isolated from cerebral cortex. Electron microscopy is used to determine synapse number and structure in vulnerable brain regions via Palkovits punch techniques as described⁶⁹. Neuronal apoptosis is quantified by TUNEL assay.

Results:

Some drug candidates target signals upstream of Tau tangle formation, reducing synaptic and neuronal damage during the development of tauopathy. These drugs inhibit early pathogenic cascades, which normally lead to memory deficit. If synapse damage and neural loss are not striking in 3×Tg, more delicate approaches are used, such as array tomography.

Effects of Drug Treatment on Cognitive Deficits in Tg Mice:

The effects of these drug candidates are tested on cognitive activity in both 3×Tg and Wt mice at age of 6 months (n=10 per group). Mice are randomly grouped and orally administered either vehicle or drug candidates at one of the two dosages (low or high) for 5 weeks. Mice completing the treatment regimen at HMRI receive 3 cognition tests: Y-maze, normal objective recognition, and Morris Water Maze.

Spatial Working Memory Y-Maze:

A Y-shape crossover design with three dark gray arms (42×4.8×20 cm) is used in the Y-maze test and novel objective recognition (NOR) tasks. Three hours after the last treatment, mice are placed at the start arm and allowed to freely explore the maze. The total number of arm entries is recorded over time. Percentage spontaneous alternation=(number of alternations)/(total arm entries−2).

Novel Objective Recognition (NOR):

Mice are habituated to the task two days before the last treatment by allowing them to explore an empty open field box (60 cm×60 cm) for 5 min. One day before the last treatment, mice after 3 hours treatment are placed in the same open field box with two identical objects in opposite corners, and allowed to freely explore. After 30 s of object exploration, the trial ends and time spent on each object is recorded. Mice that do not complete 30 s exploration within 20 min are excluded from the study.

Following the last 3 h treatment, mice are then tested in the same way with one object replaced by a novel one. Trial duration extends to 5 min. Location of the novel object (left or right side) is counterbalanced to minimize bias. A crossover design is used, with a different set of objects after a 15 day drug-free period. Discrimination index (DI) is used to evaluate the effects of drug candidates on object recognition.

DI=(time spent exploring novel object−time spent exploring familiar object)/(total time spent exploring both objects).

Reference Memory Morris Water Maze (MWM):

The reference memory version of the MWM task is performed by an experimenter blind to mouse genotype when administering DC or vehicle to Tg mice. All trials are recorded with TSE computerized video tracking system. Parameters (latency and percent of time in target quadrant) are recorded and compared with parameters from other quadrants. For the probe test, number of entries in the platform zone and time spent in target zone and in opposite quadrants is recorded.

Data Analysis:

A two-way analysis of variance (ANOVA) with genotype as the between-subject factor and treatment as the within subject factor is used for the Y-maze and object recognition tasks. Percent alternation (Y-maze) and DI (object recognition) are the dependent measures. Post hoc analyses is carried out using Bonferroni's multiple comparison tests as appropriate. Raw data that do not meet the assumption of normality and equal variance are converted using square-root transformation followed by t test. Data from MWM test is analyzed using a two-way ANOVA with genotype, day and treatment as co-variant factors. Post hoc Bonferroni analyses are conducted on significant results.

### Example 5. RNA-Seq and Canonical Pathway Analysis Shows Significant Overlap Between Clonal 3D AD Models and Human AD Patient Brains

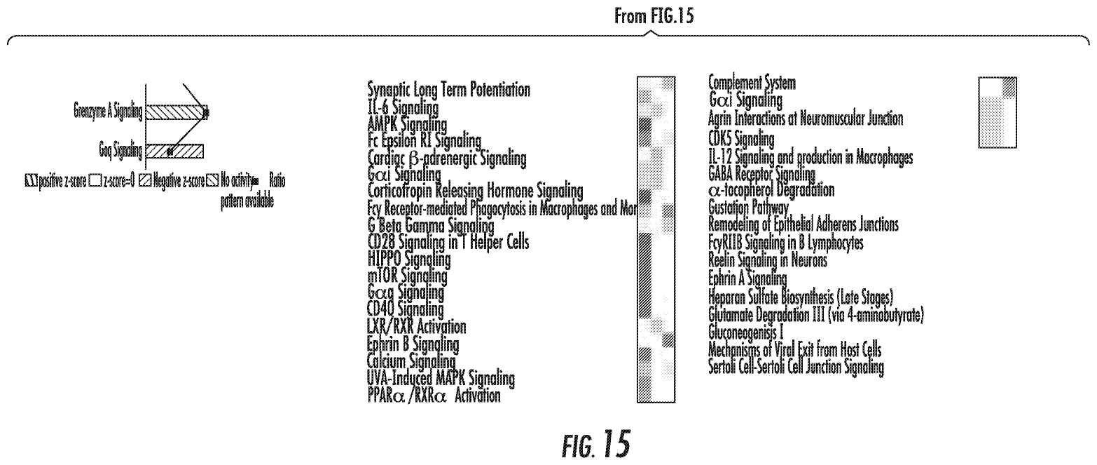

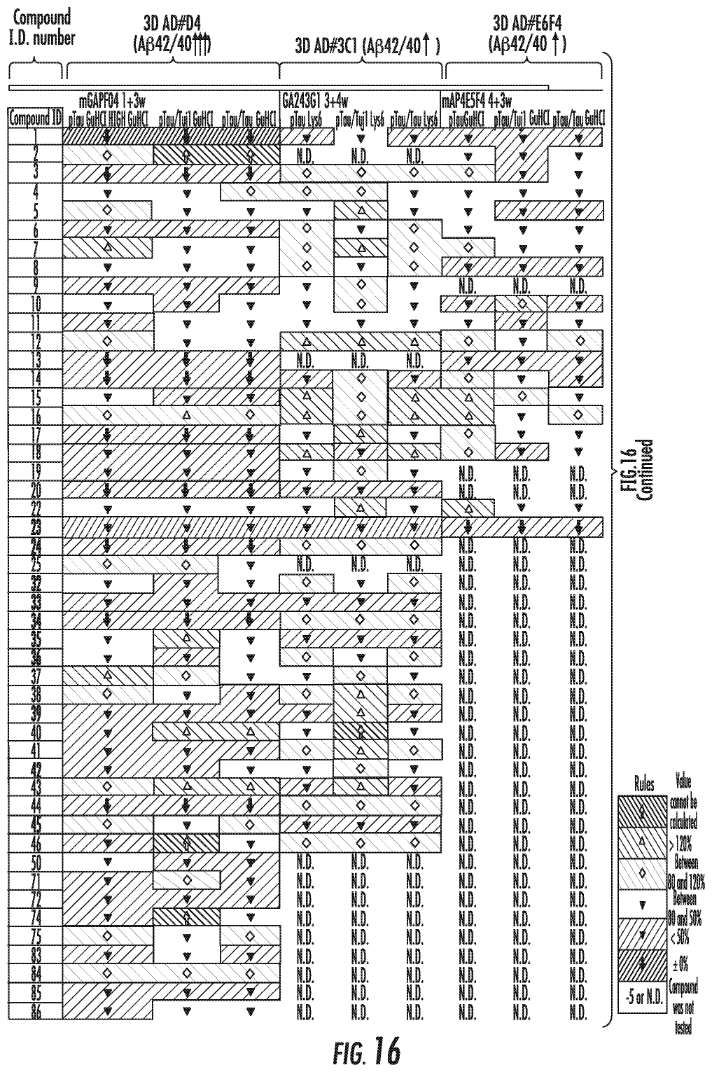

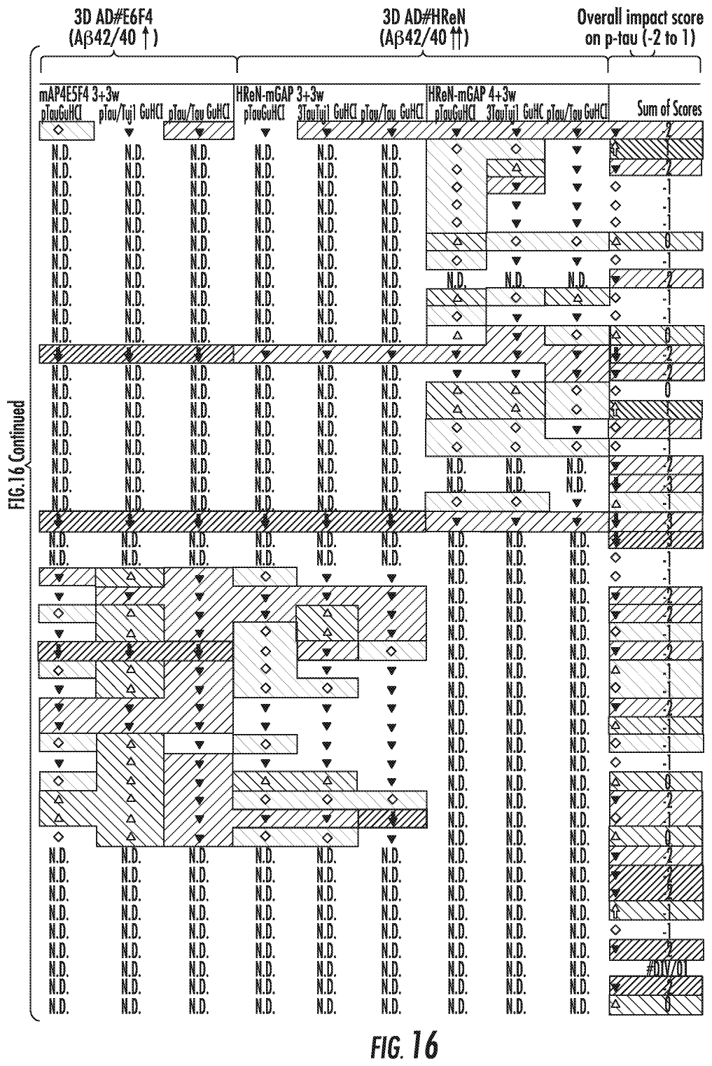

Multiple single-clonal 3D AD cell lines were used to confirm drug candidates identified from the SMART approaches. These single-clonal AD cell lines provide more reproducible results for drug screening as compared to the original mixed AD cell lines. Another advantage of using multiple single clonal lines is that the impact of candidate drugs on 3D AD models are tested with mild, moderate, or severe AD pathology. It was shown that single-clonal AD cells with higher Aβ42/40 ratio (# D4, # H10, # A4H1; FIG. 15-16) displayed robust AD pathology including pathological Aβ accumulation and insoluble aggregation of phospho- and total tau species (p-tau, t-tau), as compared to AD cells with lower Aβ42/40 ratio (# AS, #3C1; FIG. 15-16).

To examine the multiple single-clonal AD models, unbiased whole genome RNA-seq analyses were performed to compare gene expression profiles among the clonal AD models with different Aβ42/40 ratios, as compared to control 3D cultures and undifferentiated 2D control cells (FIG. 15a-d). It was found that clonal AD cell lines with different Aβ42/40 ratio (# D4, # H10, # showed distinctive differential gene expression patterns as compared to control 3D cells) (FIG. 15a). Differential gene expression profile of 3D AD cultures were analyzed after treating anti-Aβ drugs (BACE1 inhibitor, Ly2886721; Gamma-secretase modulator (GSM), GSM15606) (FIG. 15b). Canonical pathway analysis of differentially expressed genes between 3D control (G2# B2) and 3D AD model (# AS) showed significantly enriched pathways including glutamate receptor signaling, synaptic long term potentiation/depression, cAMP/CREB signaling, LPS/IL1 and RXR, which overlap with previously proposed AD pathogenic cascades. (FIG. 15c). Treatments with anti-Aβ drugs significantly altered some of these pathways (FIG. 15d). More importantly, enriched pathways were compared between the 3D AD model (# AS) and AD patient brains using available AD brain RNA-seq database.

Comparative analysis showed significant enrichment of common pathways between the 3D AD model and AD brains, including glutamate signaling, synaptic long term potentiation/depression, CREB/cAMP and Calcium signaling (FIG. 15e). These results show that this 3D AD model recapitulates AD pathogenic cascades.

### Example 6. Extensive Cross-Validation of Candidate Drugs Using Multiple Human AD Cell Lines with Different Aβ42/40 Ratios

All the primary hit candidates identified herein (from initial HCS screening and some of the additional compounds from SMART screening) were extensively cross-validated. FIG. 16 is a summary showing an example of the cross-validation approach. The impact of the compounds on insoluble p-tau (pThr181tau) and total tau levels were measured by Mesoscale ELISA (n=4 to 5) and the impact levels were summarized by coding. The summary of the effects from four clonal AD cell lines with different Aβ42/40 ratios and the overall impact scores were calculated (FIG. 16). Most of the drug candidates generally decreased insoluble p-tau levels, but some of the candidates seem to alter p-tau only in select AD lines, showing these compounds work in differential action mechanisms. More importantly, most of the identified compounds decreased p-tau levels in the severe 3D AD cells with high Aβ42/40 ratio (# D4). Similar cross-validation studies were also performed with the same cells for the impact on pathogenic Aβ species. Some of the drugs significantly decreased Aβ accumulation as well as p-tau, while most of the other candidates only decreased p-tau levels (data not shown). These results show different action mechanisms of these compounds.

### Example 7. Validation of Primary Hit Candidates Using Western Blot Analysis and Quantitative Immunofluorescence Staining in 3D AD Models with High Aβ42/40 Ratios (# HReN and # A4H1)

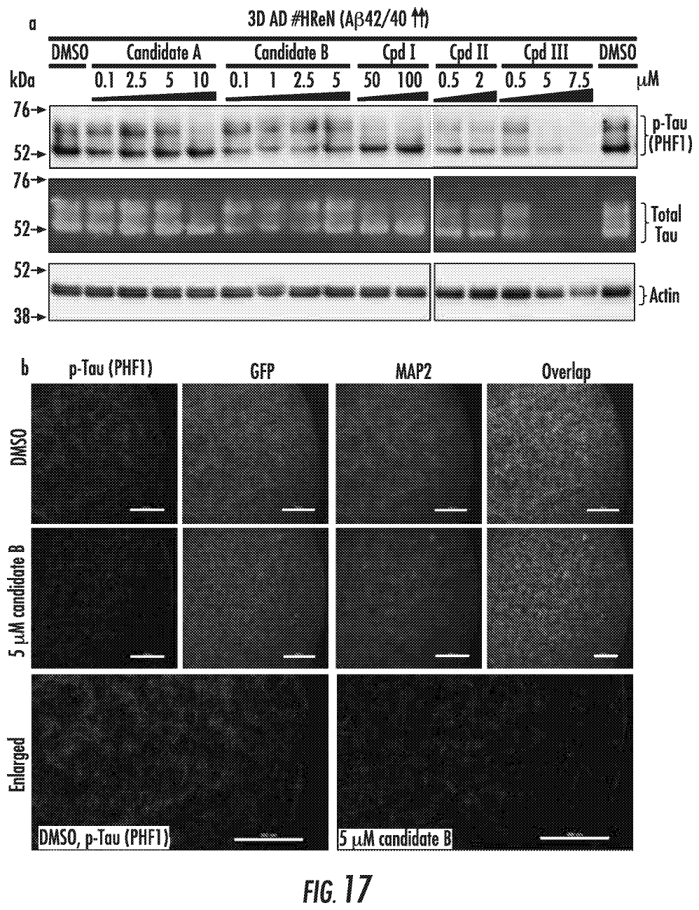

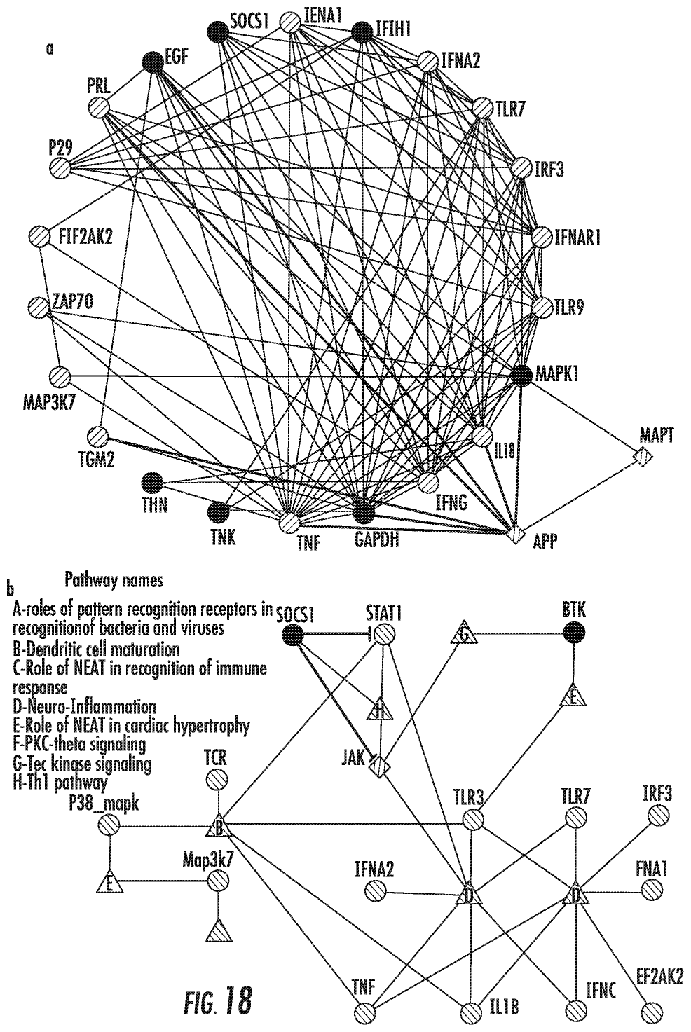

In addition to MSD Mesoscale ELISA shown in FIG. 16, quantitative Western blot and immunofluorescence analysis were used to validate candidate drugs. FIG. 17a shows Western blots further validating the impact of candidate drugs on p-tau species. Ebselen and leflunomide are compounds screened from original HCS screening of ˜24,00 biologically active/FDA-approved drug library. These compounds significantly decreased insoluble p-tau species (pSer396/Ser404, pThr181) in various concentrations (FIG. 17a). Moreover, quantitative immunofluorescence staining was used to analyze p-tau changes after treating these compounds. As shown in FIG. 17b, treatment with 5 μM leflunomide for 3 weeks robustly decreased p-tau (pSer396/Ser404) accumulation without affecting cellular viability and neurite networks.

### Example 8. Computational Modeling of RNAseq Data Reveal Possible Mechanisms Corresponding to Primary Screening Hits

The SMART framework disclosed herein can identify novel mechanisms underlying phenotypes of interest, e.g. inhibition of pTau accumulation and related pathways. Novel mechanisms identified in each round allows update on molecular signature and modification of compound ranking methods, thus generating iterative prediction-validations loops exploring different area of the searching space that might be flossed over with initial ranking strategy.

Given ebselen and leflunomide in FIG. 17, an unbiased whole genome RNAseq analysis was used to obtain transcriptomic profiles after the treatment of each compound and compare them separately to control conditions. For both treatments, a subset of genes and pathways show significant change (|log FC|>1.5) in the same direction over control condition. FIG. 18a shows a tightly-knit PPI subnetwork involving 15 down-regulated and 7 up-regulated genes after both compound treatments. These 22 genes have 102 PPI pairs among them, and there are 7 genes directly connected to APP (coding Aβ) or MAPT (coding Tau).

There are 12 down-regulated genes connected to 6 pathways, 5 of which are significantly down-regulated after treatment of both ebselen and leflunomide (FIG. 18b). It's worth noting that the enrichment of immune and inflammatory related pathway changes is consistent with the characteristics of the 3D cell model, as this system contains astrocytes, which is one of the brain innate immune cells. One of the only up-regulated genes, SOCS1, is a known suppressor for the activity of STAT-JAK pathway. Also, neuroinflammatory pathways are highly unregulated in high Abeta42/40 lines (D4 and H10) as compared to A5 (similar to GA2) (data not shown).

The thorough validation efforts using multiple human cell lines and various biochemistry and bioinformatics technologies (FIGS. 16 and 17) confirmed the ability of the SMART screening framework for identifying compounds for treating and/or preventing Alzheimer's Disease. The generation of customized RNAseq data help provide deeper insight of the similarity between the 3D cell system and AD pathology in vivo (FIG. 15), and also reveal clues for novel molecular mechanisms underlying various screening hits (FIG. 18). The generation and modeling of the RNAseq data shows the ability of the SMART framework to deal with transcriptome data generated from multiple platforms. Furthermore, FIG. 18b demonstrates that the bioinformatics methods for SMART shown herein can uncover novel mechanisms underlying pTau inhibition.

### REFERENCES CITED IN THE EXAMPLES

1. Chong C R, Sullivan D J. New uses for old drugs. Nature. 2007 Aug. 9; 448(7154):645-646.
2. Walsh D P, Chang Y-T. Chemical Genetics. Chem Rev. 2006 Jun. 1; 106(6):2476-2530.
3. Diamandis P, Wildenhain J, Clarke I D, Sacher A G, Graham J, Bellows D S, Ling E K M, Ward R J, Jamieson L G, Tyers M, Dirks P B. Chemical genetics reveals a complex functional ground state of neural stem cells. Nat Chem Biol. 2007 May; 3(5):268-273. PMID: 17417631
4. Choi S H, Kim Y H, Hebisch M, Sliwinski C, Lee S, D/'Avanzo C, Chen H, Hooli B, Asselin C, Muffat J, Klee J B, Zhang C, Wainger B J, Peitz M, Kovacs D M, Woolf C J, Wagner S L, Tanzi R E, Kim D Y. A three-dimensional human neural cell culture model of Alzheimer/'s disease. Nature. 2014 Nov. 13; 515(7526):274-278.
5. Kim Y H, Choi S H, D'Avanzo C, Hebisch M, Sliwinski C, Bylykbashi E, Washicosky K J, Klee J B, Brustle O, Tanzi R E, Kim D Y. A 3D human neural cell culture system for modeling Alzheimer's disease. Nat Protoc. 2015 July; 10(7):985-1006.
6. Oddo S, Caccamo A, Shepherd J D, Murphy M P, Golde T E, Kayed R, Metherate R, Mattson M P, Akbari Y, LaFerla F M. Triple-Transgenic Model of Alzheimer's Disease with Plaques and Tangles: Intracellular Aβ and Synaptic Dysfunction. Neuron. 2003 Jul. 31; 39(3):409-421.
7. Lamb J, Crawford E D, Peck D, Modell J W, Blat I C, Wrobel M J, Lerner J, Brunet J-P, Subramanian A, Ross K N, Reich M, Hieronymus H, Wei G, Armstrong S A, Haggarty S J, Clemons P A, Wei R, Carr S A, Lander E S, Golub T R. The Connectivity Map: Using Gene-Expression Signatures to Connect Small Molecules, Genes, and Disease. Science. 2006 Sep. 29; 313(5795):1929.
8. Lamb J. The Connectivity Map: a new tool for biomedical research. Nat Rev Cancer. 2007 January; 7(1):54-60.
9. Library of Integrated Network-based Cellular Signatures (LINCS). [Internet]. Available from: https://commonfund.nih.gov/LINCS/10.
10. Duan Q, Reid S P, Clark N R, Wang Z, Fernandez N F, Rouillard A D, Readhead B, Tritsch S R, Hodos R, Hafner M, Niepel M, Sorger P K, Dudley J T, Bavari S, Panchal R G, Ma'ayan A. L1000CDS2: LINCS L1000 characteristic direction signatures search engine. Npj Syst Biol Appl. 2016 Aug. 4; 2:16015.
11. Jin G, Fu C, Zhao H, Cui K, Chang J, Wong S T C. A novel method of transcriptional response analysis to facilitate drug repositioning for cancer therapy. Cancer Res [Internet]. 2011 Nov. 22; Available from: http://cancerres.aacrjournals.org/content/early/2011/11/21/0008-5472.CAN-11-2333.abstract
12. Zhao H, Jin G, Cui K, Ren D, Liu T, Chen P, Wong S, Li F, Fan Y, Rodriguez A, Chang J, Wong S T. Novel Modeling of Cancer Cell Signaling Pathways Enables Systematic Drug Repositioning for Distinct Breast Cancer Metastases. Cancer Res. 2013 Oct. 14; 73(20):6149.
13. Jin G, Wong S T C. Toward better drug repositioning: prioritizing and integrating existing methods into efficient pipelines. Drug Discov Today. 2014 May; 19(5):637-644.
14. Azvolinsky A. Repurposing Existing Drugs for New Indications. The Scientist. 2017 Jan. 1;
15. Choi D S, Blanco E, Kim Y-S, Rodriguez A A, Zhao H, Huang T H-M, Chen C-L, Jin G, Landis M D, Burey L A, Qian W, Granados S M, Dave B, Wong H H, Ferrari M, Wong S T C, Chang J C. Chloroquine Eliminates Cancer Stem Cells Through Deregulation of Jak2 and DNMT1. STEM CELLS. 2014 Sep. 1; 32(9):2309-2323.
16. Chloroquine With Taxane Chemotherapy for Advanced or Metastatic Breast Cancer Patients Who Have Failed an Anthracycline (CAT) [Internet]. Available from: https://clinicaltrials.gov/ct2/show/NCT01446016
17. Zhang Y, Zhou X, Witt R M, Sabatini B L, Adjeroh D, Wong S T C. Dendritic spine detection using curvilinear structure detector and LDA classifier. NeuroImage. 2007 June; 36(2):346-360.
18. Fan J, Zhou X, Dy J G, Zhang Y, Wong S T C. An Automated Pipeline for Dendrite Spine Detection and Tracking of 3D Optical Microscopy Neuron Images of In Vivo Mouse Models. Neuroinformatics. 2009; 7(2):113-130.
19. Ofengeim D, Shi P, Miao B, Fan J, Xia X, Fan Y, Lipinski M M, Hashimoto T, Polydoro M, Yuan J, Wong S T C, Degterev A. Identification of Small Molecule Inhibitors of Neurite Loss Induced by Aβ peptide using High Content Screening. J Biol Chem. 2012 Mar. 16; 287(12):8714-8723.
20. Yin Z, Zhou X, Bakal C, Li F, Sun Y, Perrimon N, Wong S T. Using iterative cluster merging with improved gap statistics to perform online phenotype discovery in the context of high-throughput RNAi screens. BMC Bioinformatics. 2008; 9(1):1-20.
21. Yin Z, Zhou X, Sun Y, Wong S T C. Online phenotype discovery based on minimum classification error model. Pattern Recognit Comput Life Sci. 2009 April; 42(4):509-522.
22. Yin Z, Sadok A, Sailem H, McCarthy A, Xia X, Li F, Garcia M A, Evans L, Barr A R, Perrimon N, Marshall C J, Wong S T C, Bakal C. A screen for morphological complexity identifies regulators of switch-like transitions between discrete cell shapes. Nat Cell Biol. 2013 July; 15(7):860-871.
23. Yin Z, Sailem H, Sero J, Ardy R, Wong S T C, Bakal C. How cells explore shape space: A quantitative statistical perspective of cellular morphogenesis. BioEssays. 2014; 36(12):1195-1203.
24. De Bondt M, van den Essen A. Singular Hessians. J Algebra. 2004 Dec. 1; 282(1):195-204.
25. Chen K, Wang Y, Yang R. Hessian matrix based saddle point detection for granules segmentation in 2D image. J Electron China. 2008; 25(6):728-736.
26. Gu X-H, Xu L-J, Liu Z-Q, Wei B, Yang Y-J, Xu G-G, Yin X-P, Wang W. The flavonoid baicalein rescues synaptic plasticity and memory deficits in a mouse model of Alzheimer's disease. Behav Brain Res. 2016 Sep. 15; 311:309-321.
27. Corbett A, Pickett J, Burns A, Corcoran J, Dunnett S B, Edison P, Hagan J J, Holmes C, Jones E, Katona C, Kearns I, Kehoe P, Mudher A, Passmore A, Shepherd N, Walsh F, Ballard C. Drug repositioning for Alzheimer's disease. Nat Rev Drug Discov. 2012 November; 11(11):833-846.
28. Liberzon A, Subramanian A, Pinchback R, Thorvaldsdóttir H, Tamayo P, Mesirov J P. Molecular signatures database (MSigDB) 3.0. Bioinformatics. 2011 May 5; 27(12):1739-1740.
29. Vidović D, Koleti A, Schürer S C. Large-scale integration of small molecule-induced genome-wide transcriptional responses, Kinome-wide binding affinities and cell-growth inhibition profiles reveal global trends characterizing systems-level drug action. Front Genet. 2014; 5:342.
30. Liu C, Su J, Yang F, Wei K, Ma J, Zhou X. Compound signature detection on LINCS L1000 big data. Mol Biosyst. 2015; 11(3):714-722.
31. Mootha V K, Lindgren C M, Eriksson K-F, Subramanian A, Sihag S, Lehar J, Puigserver P, Carlsson E, Ridderstrale M, Laurila E, Houstis N, Daly M J, Patterson N, Mesirov J P, Golub T R, Tamayo P, Spiegelman B, Lander E S, Hirschhorn J N, Altshuler D, Groop L C. PGC-1[alpha]-responsive genes involved in oxidative phosphorylation are coordinately downregulated in human diabetes. Nat Genet. 2003 July; 34(3):267-273.
32. Kanehisa M, Goto S. KEGG: Kyoto Encyclopedia of Genes and Genomes. Nucleic Acids Res. 2000 Jan. 1; 28(1):27-30.
33. Kanehisa M, Sato Y, Kawashima M, Furumichi M, Tanabe M. KEGG as a reference resource for gene and protein annotation. Nucleic Acids Res. 2015 Oct. 17; 44(D1):D457-D462.
34. Nishimura D. BioCarta. Biotech Softw Internet Rep [Internet]. 2001; 2. Available from: http://dx.doi.org/10.1089/152791601750294344
35. Biocarta pathways [Internet]. Available from: https://cgap.nci.nih.gov/Pathways/BioCarta_Pathways
36. Milacic M, Haw R, Rothfels K, Wu G, Croft D, Hermjakob H, D'Eustachio P, Stein L. Annotating Cancer Variants and Anti-Cancer Therapeutics in Reactome. Cancers. 2012; 4(4).
37. Croft D, Mundo A F, Haw R, Milacic M, Weiser J, Wu G, Caudy M, Garapati P, Gillespie M, Kamdar M R, Jassal B, Jupe S, Matthews L, May B, Palatnik S, Rothfels K, Shamovsky V, Song H, Williams M, Birney E, Hermjakob H, Stein L, D'Eustachio P. The Reactome pathway knowledgebase. Nucleic Acids Res. 2013 Nov. 15; 42(D1):D472-D477.
38. Xie X, Lu J, Kulbokas E J, Golub T R, Mootha V, Lindblad-Toh K, Lander E S, Kellis M. Systematic discovery of regulatory motifs in human promoters and 3[prime] UTRs by comparison of several mammals. Nature. 2005 Mar. 17; 434(7031):338-345.
39. KNÜPPEL R, DIETZE P, LEHNBERG W, FRECH K, WINGENDER E. TRANSFAC Retrieval Program: A Network Model Database of Eukaryotic Transcription Regulating Sequences and Proteins. J Comput Biol. 1994 Jan. 1; 1(3):191-198.
40. Ashburner M, Ball C A, Blake J A, Botstein D, Butler H, Cherry J M, Davis A P, Dolinski K, Dwight S S, Eppig J T, Harris M A, Hill D P, Issel-Tarver L, Kasarskis A, Lewis S, Matese J C, Richardson J E, Ringwald M, Rubin G M, Sherlock G. Gene Ontology: tool for the unification of biology. Nat Genet. 2000 May; 25(1):25-29.
41. Gene Ontology Consortium: going forward. Nucleic Acids Res. 2014 Nov. 26; 43(D1):D1049-D1056.
42. Liberzon A, Birger C, Thorvaldsdóttir H, Ghandi M, Mesirov J P, Tamayo P. The Molecular Signatures Database Hallmark Gene Set Collection. Cell Syst. 2015 Dec. 23; 1(6):417-425.
43. Iorio F, Bosotti R, Scacheri E, Belcastro V, Mithbaokar P, Ferriero R, Murino L, Tagliaferri R, Brunetti-Pierri N, Isacchi A, di Bernardo D. Discovery of drug mode of action and drug repositioning from transcriptional responses. Proc Natl Acad Sci. 2010 Aug. 17; 107(33):14621-14626.
44. Kuhn M, Szklarczyk D, Franceschini A, von Mering C, Jensen L J, Bork P. STITCH 3: zooming in on protein-chemical interactions. Nucleic Acids Res. 2011 Nov. 9; 40(D1):D876-D880.
45. Martin Y C, Kofron J L, Traphagen L M. Do Structurally Similar Molecules Have Similar Biological Activity? J Med Chem. 2002 Sep. 1; 45(19):4350-4358.
46. Online Mendelian Inheritance in Man, OMIM® [Internet]. McKusick-Nathans Institute of Genetic Medicine, Johns Hopkins University (Baltimore, Md.); Available from: https://omim.org/
47. Jensen L J, Saric J, Bork P. Literature mining for the biologist: from information retrieval to biological discovery. Nat Rev Genet. 2006 February; 7(2):119-129.
48. S̆arić J, Jensen L J, Ouzounova R, Rojas I, Bork P. Extraction of regulatory gene/protein networks from Medline. Bioinformatics. 2005 Jul. 26; 22(6):645-650.
49. Bang-Jensen J, Gutin G. Directed graphs: Theory, Algorithms and Applications, 2nd edition. Springer; 2009.
50. You Z-H, Yin Z, Han K, Huang D-S, Zhou X. A semi-supervised learning approach to predict synthetic genetic interactions by combining functional and topological properties of functional gene network. BMC Bioinformatics. 2010; 11(1):343.
51. Barrat A, Barthelemy M, Pastor-Satorras R, Vespignani A. The architecture of complex weighted networks. Proc Natl Acad Sci USA [Internet]. 2004; 101. Available from: http://dx.doi.org/10.1073/pnas.0400087101
52. Stephenson K, Zelen M. Rethinking Centrality: Methods and Applications. Soc Netw [Internet]. 1989; 11. Available from: http://dx.doi.org/10.1016/0378-8733(89)90016-6
53. Brandes U, Fleischer D. Centrality measures based on current flow. Stacs 2005 Proc [Internet]. 2005; 3404. Available from: http://dx.doi.org/10.1007/978-3-540-31856-9_44
54. Cortes C, Vapnik V. Support-Vector Networks. Mach Learn. 1995; 20.
55. Chang C-. C, Lin C-. J. LIBSVM Libr Support Vector Mach. 2001.
56. Guyon I, Weston J, Barnhill S, Vapnik V. Gene Selection for Cancer Classification using Support Vector Machines. Mach Learn. 2002; 46(1):389-422.
57. Globally Harmonized System of Classification and Labelling of Chemicals (GHS), Rev. 6 [Internet]. United Nations; 2015. Available from: http://www.unece.org/trans/danger/publi/ghs/ghs_rev06/06files_e.html#c38156
58. Bhattacharya A, De R K. Divisive Correlation Clustering Algorithm (DCCA) for grouping of genes: detecting varying patterns in expression profiles. Bioinformatics [Internet]. 2008; 24. Available from: http://dx.doi.org/10.1093/bioinformatics/btn133
59. Lee J-H, Kim D G, Bae T J, Rho K, Kim J-T, Lee J-J, Jang Y, Kim B C, Park K M, Kim S. CDA: Combinatorial Drug Discovery Using Transcriptional Response Modules. PLOS ONE. 2012 Aug. 8; 7(8):e42573.
60. Huang L, Li F, Sheng J, Xia X, Ma J, Zhan M, Wong S T C. DrugComboRanker: drug combination discovery based on target network analysis. Bioinformatics. 2014 Jun. 11; 30(12):i228-i236.
61. Eisen M B, Spellman P T, Brown P O, Botstein D. Cluster analysis and display of genome-wide expression patterns. Proc Natl Acad Sci USA [Internet]. 1998; 95. Available from: http://dx.doi.org/10.1073/pnas.95.25.14863
62. Reich M, Liefeld T, Gould J, Lerner J, Tamayo P, Mesirov J P. GenePattern 2.0. Nat Genet. 2006 May; 38(5):500-501.
63. Opsahl T, Panzarasa P. Clustering in weighted networks. Soc Netw [Internet]. 2009; 31. Available from: http://dx.doi.org/10.1016/j.socnet.2009.02.002
64. Hinton G E, Osindero S, Teh Y-W. A Fast Learning Algorithm for Deep Belief Nets. Neural Comput. 2006 May 17; 18(7):1527-1554.
65. Subramanian A, Tamayo P, Mootha V K, Mukherjee S, Ebert B L, Gillette M A, Paulovich A, Pomeroy S L, Golub T R, Lander E S, Mesirov J P. Gene set enrichment analysis: A knowledge-based approach for interpreting genome-wide expression profiles. Proc Natl Acad Sci. 2005 Oct. 25; 102(43):15545-15550.
66. Gilks W R, Best N G, Tan K K C. Adaptive Rejection Metropolis Sampling within Gibbs Sampling. J R Stat Soc Ser C Appl Stat. 1995; 44(4):455-472.
67. Meyer R, Cai B, Perron F. Adaptive rejection Metropolis sampling using Lagrange interpolation polynomials of degree 2. Comput Stat Data Anal. 2008 Mar. 15; 52(7):3408-3423.
68. D'Avanzo C, Aronson J, Kim Y H, Choi S H, Tanzi R E, Kim D Y. Alzheimer's in 3D culture: Challenges and perspectives. BioEssays. 2015 Oct. 1; 37(10):1139-1148.
69. Xie W, Li X, Li C, Zhu W, Jankovic J, Le W. Proteasome inhibition modeling nigral neuron degeneration in Parkinson's disease. J Neurochem. 2010 Oct. 1; 115(1):188-199.
70. Dunkley P R, Jarvie P E, Robinson P J. A rapid Percoll gradient procedure for preparation of synaptosomes. Nat Protoc. 2008 October; 3(11):1718-1728.
71. Galli S, Lopes D M, Amman R, Kopra J, Millar S E, Gibb A, Salinas P C. Deficient Wnt signalling triggers striatal synaptic degeneration and impaired motor behaviour in adult mice. Nat Commun. 2014 Oct. 16; 5:4992.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

We claim:

1. A method for treating or preventing Alzheimer's disease comprising administering to a subject in need thereof a therapeutically effective amount of a compound selected from the following: 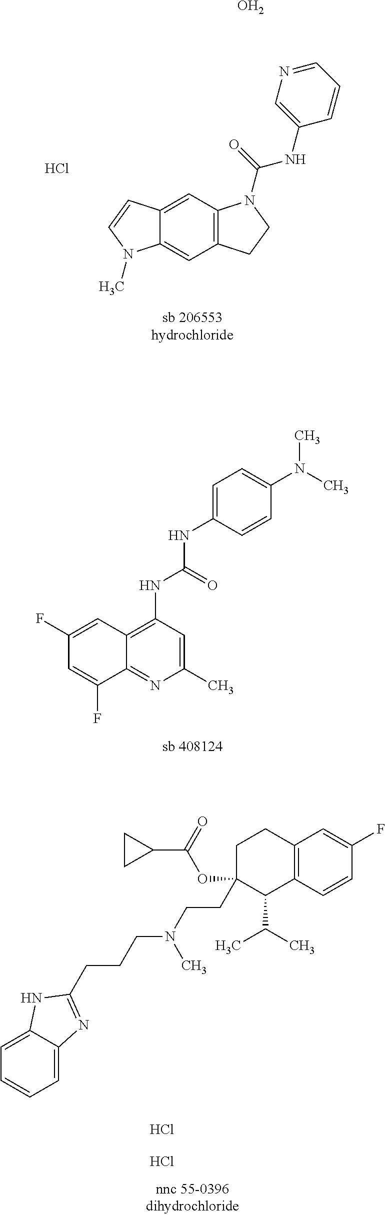 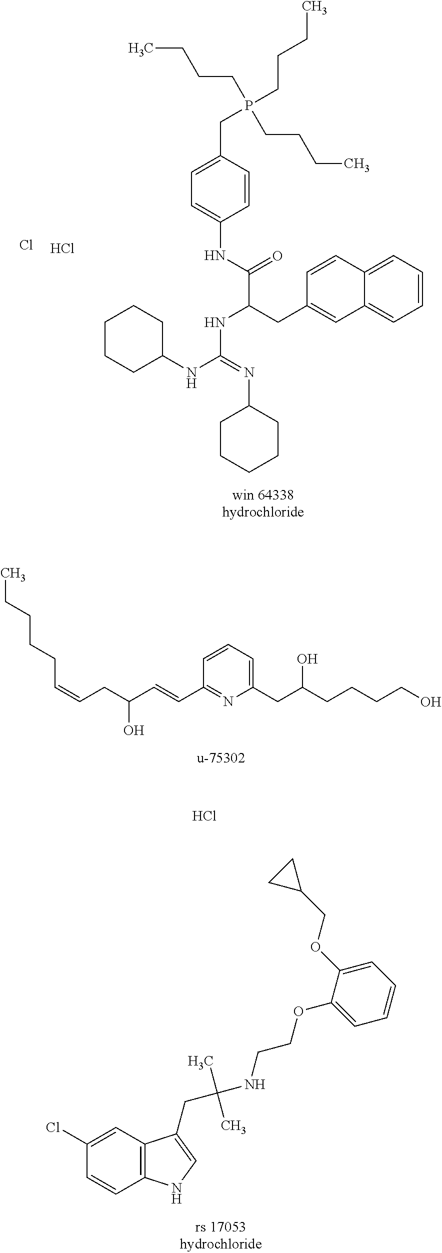 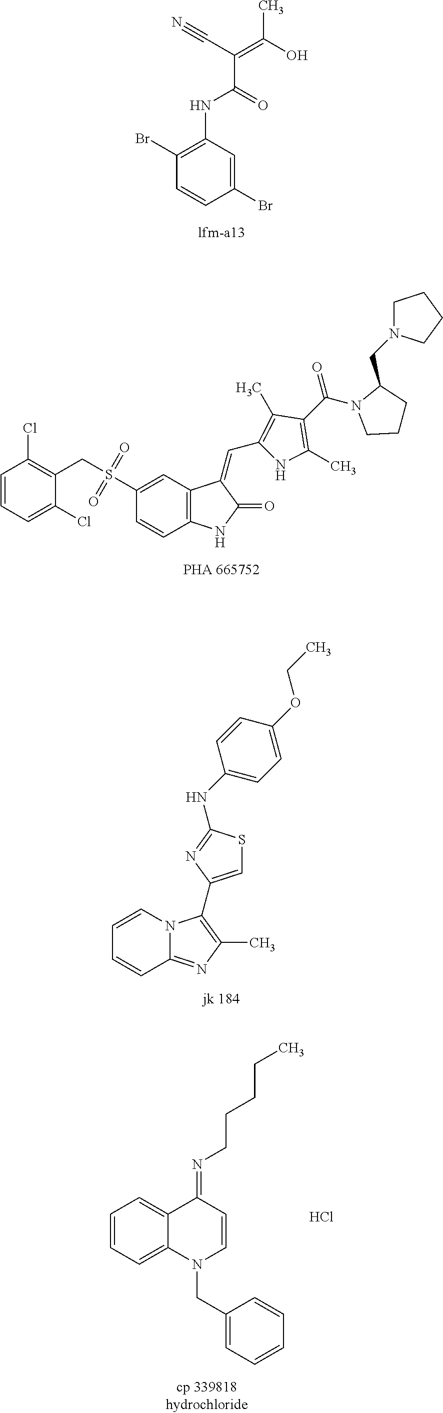 [SMILES: C/C(O)=C(\C#N)C(=O)Nc1cc(Br)ccc1Br ; Cc1[nH]c(/C=C2\C(=O)Nc3ccc(S(=O)(=O)Cc4c(Cl)cccc4Cl)cc32)c(C)c1C(=O)N1CCC[C@@H]1CN1CCCC1 ; CCOc1ccc(Nc2nc(-c3c(C)nc4ccccn34)cs2)cc1 ; CCCCC/N=c1\ccn(Cc2ccccc2)c2ccccc12 ; Cl] 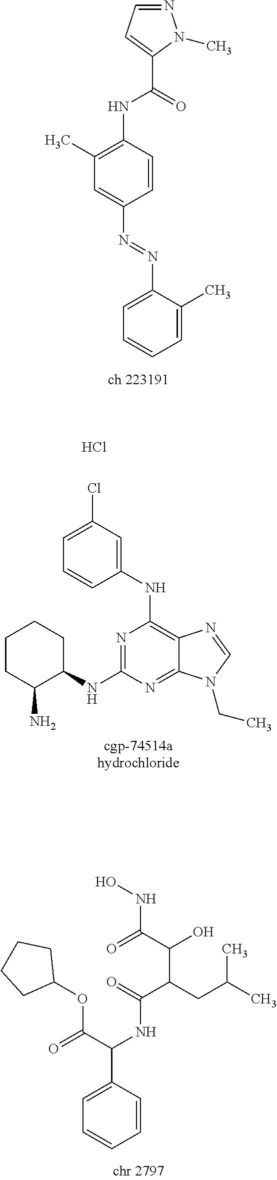 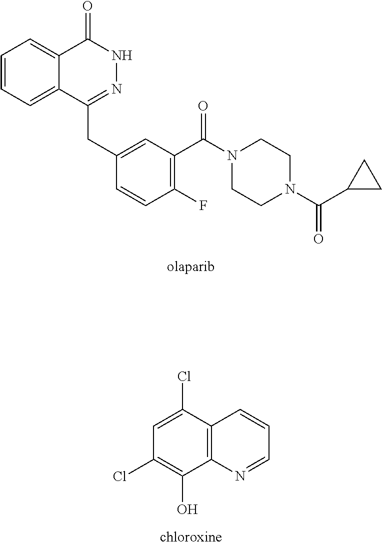 [SMILES: O=C(c1cc(Cc2n[nH]c(=O)c3ccccc23)ccc1F)N1CCN(C(=O)C2CC2)CC1 ; Oc1c(Cl)cc(Cl)c2cccnc12]
or a pharmaceutically acceptable salt thereof.

2. The method of claim 1, wherein the compound is sb 206553 hydrochloride.

3. The method of claim 1, wherein the compound is sb 408124.

4. The method of claim 1, wherein the compound is nnc 55-0396 dihydrochloride.

5. The method of claim 1, wherein the compound is win 64338 hydrochloride.

6. The method of claim 1, wherein the compound is u-75302.

7. The method of claim 1, wherein the compound is rs 17053 hydrochloride.

8. The method of claim 1, wherein the compound is lfm-a13.

9. The method of claim 1, wherein the compound is PHA 665752.

10. The method of claim 1, wherein the compound is jk 184.

11. The method of claim 1, wherein the compound is cp 339818 hydrochloride.

12. The method of claim 1, wherein the compound is ch 223191.

13. The method of claim 1, wherein the compound is cgp-74514a hydrochloride.

14. The method of claim 1, wherein the compound is chr 2797.

15. The method of claim 1, wherein the compound is olaparib.

16. The method of claim 1, wherein the compound is chloroxine.

17. The method of claim 1, further comprising administering to the subject an additional therapeutic agent.

18. The method of claim 17, wherein the additional therapeutic agent is selected from memantine, donepezil, galantamine, tacrine hydrochloride, and rivastigmine tartrate.

19. A method for inhibiting tau phosphorylation comprising administering to a subject a compound selected from the following: 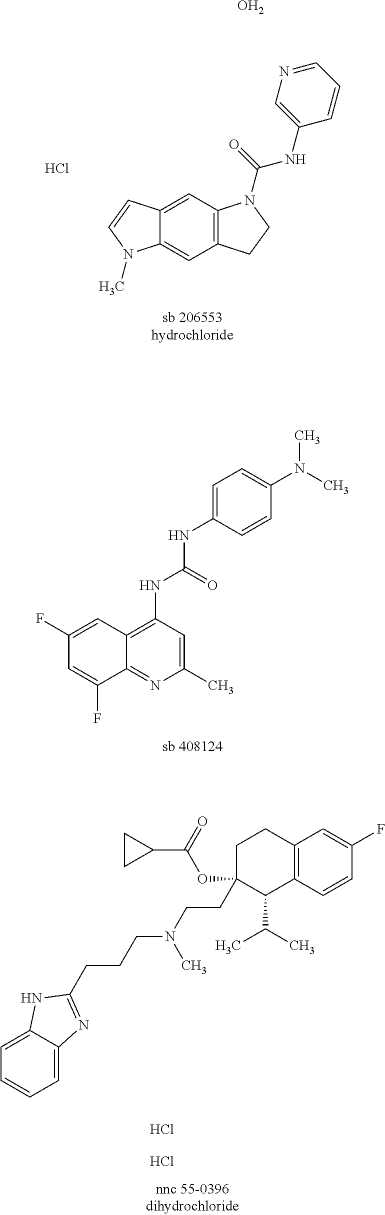 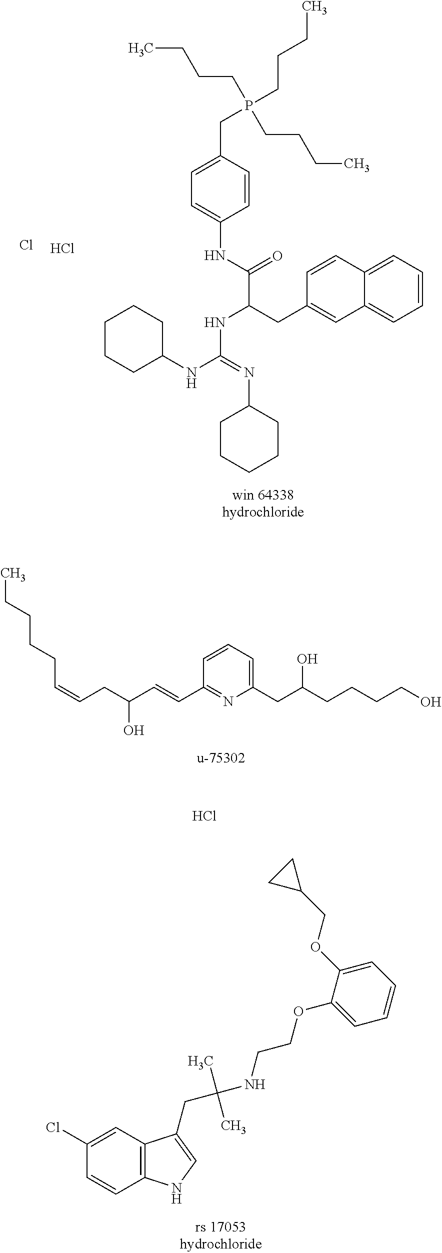 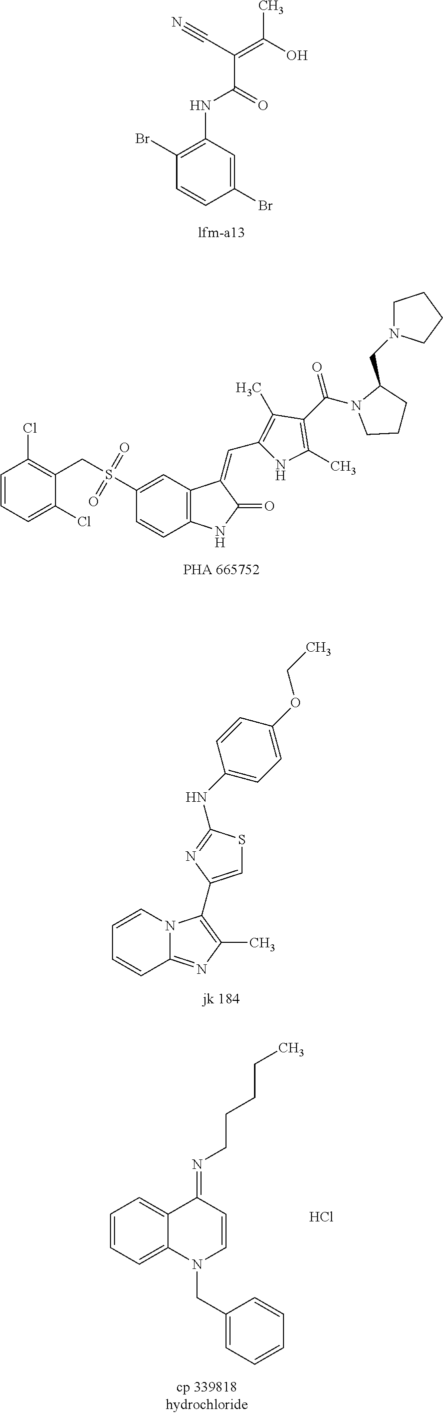 [SMILES: C/C(O)=C(\C#N)C(=O)Nc1cc(Br)ccc1Br ; Cc1[nH]c(/C=C2\C(=O)Nc3ccc(S(=O)(=O)Cc4c(Cl)cccc4Cl)cc32)c(C)c1C(=O)N1CCC[C@@H]1CN1CCCC1 ; CCOc1ccc(Nc2nc(-c3c(C)nc4ccccn34)cs2)cc1 ; CCCCC/N=c1\ccn(Cc2ccccc2)c2ccccc12 ; Cl] 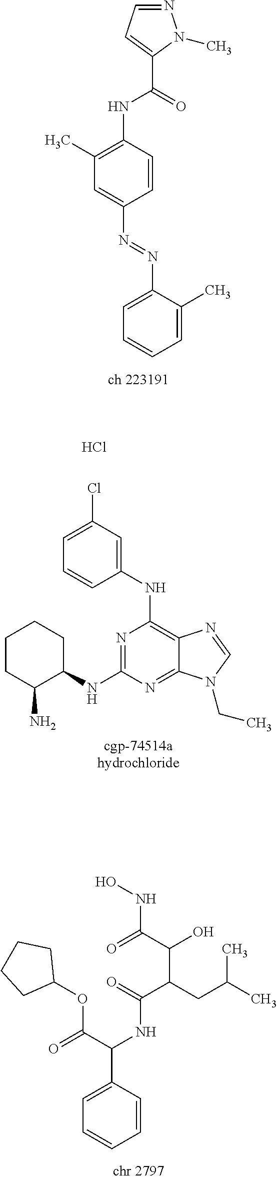 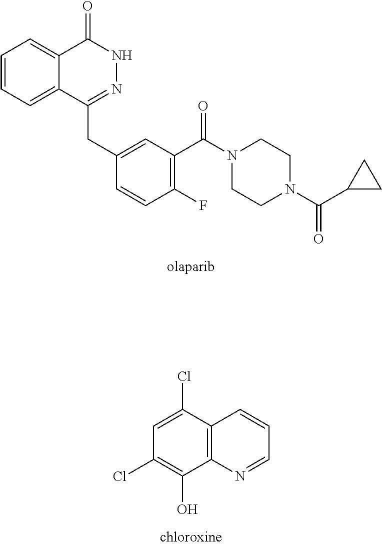 [SMILES: O=C(c1cc(Cc2n[nH]c(=O)c3ccccc23)ccc1F)N1CCN(C(=O)C2CC2)CC1 ; Oc1c(Cl)cc(Cl)c2cccnc12]
or a pharmaceutically acceptable salt thereof.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. A method for inhibiting tau phosphorylation comprising administering to a subject a compound selected from the following: 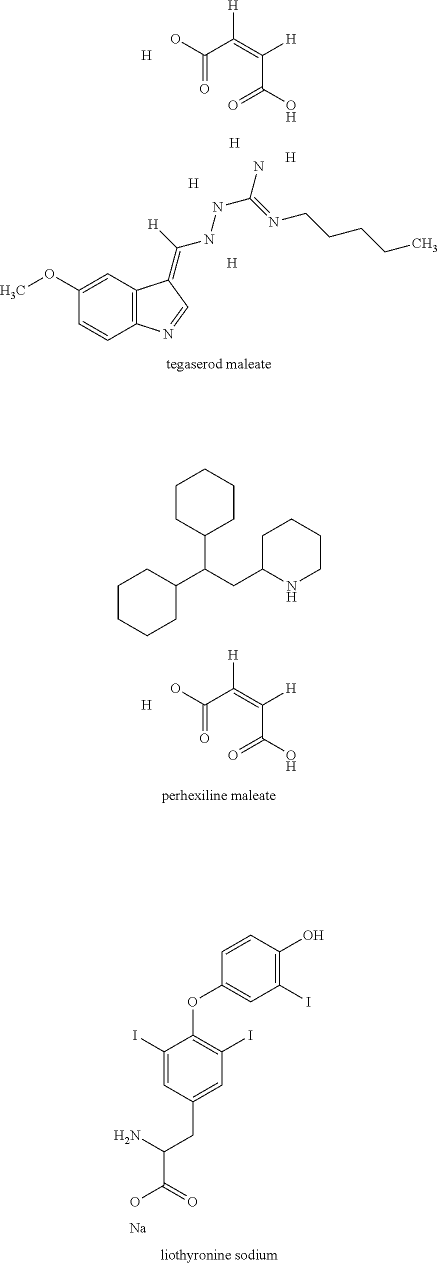 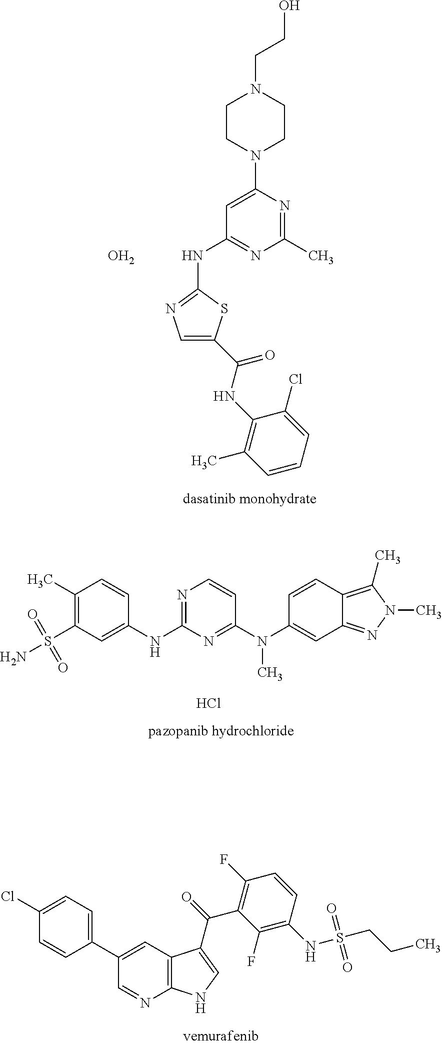 [SMILES: O ; Cc1nc(Nc2ncc(C(=O)Nc3c(C)cccc3Cl)s2)cc(N2CCN(CCO)CC2)n1 ; Cc1ccc(Nc2nccc(N(C)c3ccc4c(C)n(C)nc4c3)n2)cc1S(N)(=O)=O ; Cl ; CCCS(=O)(=O)Nc1ccc(F)c(C(=O)c2c[nH]c3ncc(-c4ccc(Cl)cc4)cc23)c1F] 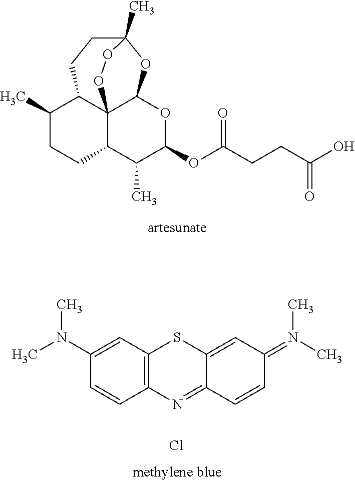
or a pharmaceutically acceptable salt thereof.

38. (canceled)

39. (canceled)

40. (canceled)

41. (canceled)

42. (canceled)

43. (canceled)

44. (canceled)

45. (canceled)

46. (canceled)

47. (canceled)
